SEQUENCE LISTING (45 sequences)

SEQ ID NO: 1 - protein, 10 aa, Artificial
  Synthetic construct
AYTISRNSMG

SEQ ID NO: 2 - protein, 10 aa, Artificial
  Synthetic construct
ADPMSRNSMG

SEQ ID NO: 3 - protein, 10 aa, Artificial
  Synthetic construct
DNPMSRNSAG

SEQ ID NO: 4 - protein, 9 aa, Artificial
  Synthetic construct
AIESDGSTS

SEQ ID NO: 5 - protein, 24 aa, Artificial
  Synthetic construct
PKVGLGPRTALGHLAFMTLPALNY

SEQ ID NO: 6 - protein, 24 aa, Artificial
  Synthetic construct
PKVGLGPRTALGHLAFMTLQDLND

SEQ ID NO: 7 - protein, 24 aa, Artificial
  Synthetic construct
PKVGLGPRTALGHLAFMILQDLNY

SEQ ID NO: 8 - protein, 24 aa, Artificial
  Synthetic construct
PKVGLGPRTALGHLAFMILADLND

SEQ ID NO: 9 - protein, 25 aa, Artificial
  Synthetic construct
QVQLQESGGGLVQPGGSLRLSCAAS

SEQ ID NO: 10 - protein, 14 aa, Artificial
  Synthetic construct
WFRQAPGKGLEGVA

SEQ ID NO: 11 - protein, 39 aa, Artificial
  Synthetic construct
YSDSVKGRFTISLDNSKNTLYLEMNSLRAEDTAVYYCAA

SEQ ID NO: 12 - protein, 11 aa, Artificial
  Synthetic construct
WGQGTLVTVSS

SEQ ID NO: 13 - protein, 132 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMTLPALNYWGQGTLVTVSS

SEQ ID NO: 14 - protein, 132 aa, Artificial
  Synthetic construct
QVQLQESGGGLVQPGGSLRLSCAASADPMSRNSMGWFRQAPGKGLEGVAAIESDGSTSYSDSVKGRFTISLDNSKNTLYLEMNSLRAEDTAVYYCAAPKVGLGPRTALGHLAFMTLPALNYWGQGTLVTVSS

SEQ ID NO: 15 - protein, 132 aa, Artificial
  Synthetic construct
QVQLQESGGGLVQPGGSLRLSCAASDNPMSRNSAGWFRQAPGKGLEGVAAIESDGSTSYSDSVKGRFTISLDNSKNTLYLEMNSLRAEDTAVYYCAAPKVGLGPRTALGHLAFMTLPALNYWGQGTLVTVSS

SEQ ID NO: 16 - protein, 132 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMTLQDLNDWGQGTLVTVSS

SEQ ID NO: 17 - protein, 132 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMILQDLNYWGQGTLVTVSS

SEQ ID NO: 18 - protein, 132 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMILADLNDWGQGTLVTVSS

SEQ ID NO: 19 - protein, 359 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMTLPALNYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 359 aa, Artificial
  Synthetic construct
QVQLQESGGGLVQPGGSLRLSCAASADPMSRNSMGWFRQAPGKGLEGVAAIESDGSTSYSDSVKGRFTISLDNSKNTLYLEMNSLRAEDTAVYYCAAPKVGLGPRTALGHLAFMTLPALNYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 359 aa, Artificial
  Synthetic construct
QVQLQESGGGLVQPGGSLRLSCAASDNPMSRNSAGWFRQAPGKGLEGVAAIESDGSTSYSDSVKGRFTISLDNSKNTLYLEMNSLRAEDTAVYYCAAPKVGLGPRTALGHLAFMTLPALNYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 359 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMTLQDLNDWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 359 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMILQDLNYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 359 aa, Artificial
  Synthetic construct
QVQLQESGGGLVQPGGSLRLSCAASAYTISRNSMGWFRQAPGKGLEGVAAIESDGSTSYSDSVKGRFTISLDNSKNTLYLEMNSLRAEDTAVYYCAAPKVGLGPRTALGHLAFMILADLNDWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 227 aa, Artificial
  Synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 4827 bp, Artificial
  Synthetic construct
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattccctacttcatacattttcaattaagatgcagttacttcgctgtttttcaatattttctgttattgcttcagttttagcaggatcctgacatagtagggattataaggaggcggtggatccgattacaaggatgacgatgacaagggcggaggaggctcccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacgactagcaaaggcagccccataaacacacagtatgttttttaatgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 27 - DNA, 47 bp, Artificial
  Synthetic construct
catgttgtttacggagaaatgaaaagtatattgtattttgtacgagc

SEQ ID NO: 28 - DNA, 27 bp, Artificial
  Synthetic construct
cggtttgtattacttcttattcaaatg

SEQ ID NO: 29 - DNA, 43 bp, Artificial
  Synthetic construct
gttattgcttcagttttagcacaggtgcagctgcaggaatcag

SEQ ID NO: 30 - DNA, 59 bp, Artificial
  Synthetic construct
gtcatcgtcatccttgtaatcggatccaccgcctccgctagacactgtcacgagggttc

SEQ ID NO: 31 - DNA, 72 bp, Artificial
  Synthetic construct
  misc_feature (23)..(24): n is a, c, g, or t
  misc_feature (26)..(27): n is a, c, g, or t
  misc_feature (29)..(30): n is a, c, g, or t
  misc_feature (32)..(33): n is a, c, g, or t
  misc_feature (35)..(36): n is a, c, g, or t
  misc_feature (38)..(39): n is a, c, g, or t
  misc_feature (41)..(42): n is a, c, g, or t
  misc_feature (44)..(45): n is a, c, g, or t
  misc_feature (47)..(48): n is a, c, g, or t
gagactgtcttgcgccgccagcnnknnknnknnknnknnknnknnknnkggttggttcagacaggccccagg

SEQ ID NO: 32 - DNA, 63 bp, Artificial
  Synthetic construct
  misc_feature (21)..(22): n is a, c, g, or t
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
  misc_feature (36)..(37): n is a, c, g, or t
  misc_feature (39)..(40): n is a, c, g, or t
gactggaaggagtggcagctnnknnknnknnknnknnknnkagctacagcgatagcgtgaagg

SEQ ID NO: 33 - DNA, 64 bp, Artificial
  Synthetic construct
  misc_feature (21)..(22): n is a, c, g, or t
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
  misc_feature (36)..(37): n is a, c, g, or t
  misc_feature (39)..(40): n is a, c, g, or t
  misc_feature (42)..(43): n is a, c, g, or t
ccgtgtactattgcgccgctnnknnknnknnknnknnknnknnkaccgctctgggacatctggc

SEQ ID NO: 34 - DNA, 67 bp, Artificial
  Synthetic construct
  misc_feature (23)..(24): n is a, c, g, or t
  misc_feature (26)..(27): n is a, c, g, or t
  misc_feature (29)..(30): n is a, c, g, or t
  misc_feature (32)..(33): n is a, c, g, or t
  misc_feature (35)..(36): n is a, c, g, or t
  misc_feature (38)..(39): n is a, c, g, or t
  misc_feature (41)..(42): n is a, c, g, or t
  misc_feature (44)..(45): n is a, c, g, or t
caaagtgggactgggacctagannknnknnknnknnknnknnknnkatgaccctgccagcactgaac

SEQ ID NO: 35 - DNA, 66 bp, Artificial
  Synthetic construct
  misc_feature (22)..(23): n is a, c, g, or t
  misc_feature (25)..(26): n is a, c, g, or t
  misc_feature (28)..(29): n is a, c, g, or t
  misc_feature (31)..(32): n is a, c, g, or t
  misc_feature (34)..(35): n is a, c, g, or t
  misc_feature (37)..(38): n is a, c, g, or t
  misc_feature (40)..(41): n is a, c, g, or t
  misc_feature (43)..(44): n is a, c, g, or t
gctctgggacatctggccttcnnknnknnknnknnknnknnknnktggggccagggaaccctcgtg

SEQ ID NO: 36 - DNA, 22 bp, Artificial
  Synthetic construct
gctggcggcgcaagacagtctc

SEQ ID NO: 37 - DNA, 20 bp, Artificial
  Synthetic construct
agctgccactccttccagtc

SEQ ID NO: 38 - DNA, 20 bp, Artificial
  Synthetic construct
agcggcgcaatagtacacgg

SEQ ID NO: 39 - DNA, 22 bp, Artificial
  Synthetic construct
tctaggtcccagtcccactttg

SEQ ID NO: 40 - DNA, 21 bp, Artificial
  Synthetic construct
gaaggccagatgtcccagagc

SEQ ID NO: 41 - protein, 449 aa, Artificial
  Synthetic construct
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, Artificial
  Synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQADLPAFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 24 aa, Artificial
  Synthetic construct
METDTLLLWVLLLWVPGSTGGSTG

SEQ ID NO: 44 - protein, 10 aa, Artificial
  Synthetic construct
  misc_feature: Xaa can be T or P
  misc_feature (1)..(1): Xaa can be A or D
  misc_feature (2)..(2): Xaa can be Y or D
  misc_feature (3)..(3): Xaa can be any naturally occurring amino acid
  misc_feature (4)..(4): Xaa can be I or M
  misc_feature (9)..(9): Xaa can be M or A
XXXXSRNSXG

SEQ ID NO: 45 - protein, 26 aa, Artificial
  Synthetic construct
  misc_feature (20)..(20): Xaa can be T or I
  misc_feature (22)..(22): Xaa can be P, Q or A
  misc_feature (23)..(23): Xaa can be A or D
  misc_feature (26)..(26): Xaa can be Y or D
AAPKVGLGPRTALGHLAFMXLXXLNX

(19) United States
(12) Patent Application Publication
(10) Pub. No.: US 2022/0127364 A1
(43) Pub. Date: Apr. 28, 2022

(54) NOVEL ANTI-PD-L1 ANTIBODY AND USES THEREOF

(71) Applicant: INNOVENT BIOLOGICS (SUZHOU) CO., LTD., Suzhou, Jiangsu (CN)

(72) Inventors: Chunyin GU, Suzhou, Jiangsu (CN); Qian ZHOU, Suzhou, Jiangsu (CN); Zhihui KUANG, Suzhou, Jiangsu (CN); Junjian LIU, Suzhou, Jiangsu (CN)

(21) Appl. No.: 17/427,447

(22) PCT Filed: Jan. 31, 2020
(86) PCT No.: PCT/CN2020/074095

§ 371 Date: Jul. 30, 2021

(30) Foreign Application Priority Data
Feb. 1, 2019 (CN) 201910101890.8

(51) Int. Cl. C07K 16/28; A61K 45/06; A61K 39/395; G01N 33/68

(52) U.S. Cl. CPC C07K 16/2827; A61K 45/06; A61K 39/3955; G01N 33/68; G01N 2333/70532; C07K 2317/92; C07K 2317/76; C07K 2317/31; C07K 2317/569

(57) ABSTRACT

The present invention relates to a novel single-domain antibody and a fragment thereof that specifically bind to PD-L1 and a composition comprising the antibody or the fragment thereof. In addition, the present invention relates to a nucleic acid encoding the antibody or the fragment thereof, a host cell comprising the nucleic acid, and related uses. Furthermore, the present invention relates to therapeutic and diagnostic uses of the antibody and the fragment thereof. In particular, the present invention relates to combination therapies of these antibodies and the fragments thereof with other therapies, such as therapeutic modalities or therapeutic agents.

The present invention relates to a novel antibody and a fragment thereof that specifically bind to PD-L1 and a composition comprising the antibody or the fragment thereof. In addition, the present invention relates to a nucleic acid encoding the antibody or the fragment thereof, a host cell comprising the nucleic acid, and related uses. Furthermore, the present invention relates to therapeutic and diagnostic uses of the antibody and the fragment thereof, and particularly to combination therapies of these antibodies and the fragments thereof with other therapies, such as therapeutic modalities or therapeutic agents.

### BACKGROUND

Programmed death ligand 1 (PD-L1) is a protein involved in suppressing immune system responses during infections, pregnancy, tissue allografts, autoimmune diseases, and cancer. PD-L1 regulates immune responses by binding to an inhibitory receptor called programmed death 1 (PD-1) which is expressed on the surface of T cells, B cells, and monocytes. PD-L1 also negatively regulates T cell functions through interaction with another receptor B7.1 (also known as B7-1 or CD80). The formation of PD-L1/PD-1 and PD-L1/B7.1 complexes negatively regulate T cell receptor signaling, leading to subsequent down regulation of T cell activation and inhibition of anti-tumor immune activity. PD-L1 is overexpressed in many cancers. PD-L1 overexpression in tumor cells could promote tumor invasion and is often associated with poor prognosis.

The successful application of monoclonal antibodies in cancer detection and biological targeted therapy has led to a revolution in tumor therapy. However, the conventional monoclonal antibody has too high molecular weight (150 kD) and is difficult to penetrate tissues, so that the effective concentration of a tumor region is low, and the treatment effect is insufficient; the traditional antibody has high immunogenicity, and the modified antibody has difficulty in achieving the original affinity. In addition, the clinical application and popularization of the conventional fully humanized antibody are limited by many factors such as a long development cycle, high production costs, and insufficient stability. Therefore, there is a need to develop new antibody molecules with low molecular weight.

A single-domain antibody is the smallest antibody molecule available today with a molecular weight being 1/10 of that of a common antibody. Besides the antigen reactivity of the monoclonal antibody, the single-domain antibody has some unique functional characteristics, such as low molecular weight, strong stability, good solubility, easy expression, weak immunogenicity, strong penetrability, strong targeting property, simple humanization, low preparation costs and the like, and almost overcomes the defects of long development cycle, low stability, harsh storage conditions and the like of the conventional antibody.

Therefore, there is an urgent need in the art to develop a novel antibody against PD-L1, which has higher affinity than known single-domain antibodies, a lower dissociation rate after binding to PD-L1 and an improved expression yield, and thereby is more favorable for subsequent production and drug development.

### BRIEF SUMMARY

The present invention thus discloses a novel antibody molecule binding to PD-L1, such as a heavy-chain antibody molecule or a single-domain antibody molecule.

In some embodiments, the antibody or the fragment thereof of the present invention (specifically) binds to PD-L1. In some embodiments, the antibody or the fragment thereof of the present invention (specifically) binds to human PD-L1.

In some embodiments, the expression of the antibody of the present invention is far greater than that of known PD-L1 antibodies (e.g., CN 107686520 A, a single-domain antibody shown in SEQ ID NO: 14, or corresponding heavy-chain antibody thereof), and, e.g., is at least 10, 11, 12, 13, 14, 15, 16, 17, 18, 19, 20, 21, or 22-fold the expression yield of the known PD-L1 or higher under the same conditions.

In some embodiments, the anti-PD-L1 antibody or the fragment thereof of the present invention binds to PD-L1 (e.g., human PD-L1) with high affinity, for example, binds to PD-L1 with an equilibrium dissociation constant (K_{D}) of less than or equal to about 40 nM, preferably less than or equal to about 30 nM or 20 nM, more preferably less than or equal to about 15 nM, and more preferably less than or equal to about 10 nM, 9 nM, 8 nM, 7 nM, 6 nM, 5 nM, or 4.5 nM. In some embodiment, the K_{D} is above about 3 nM, 3.5 nM, or 4 nM, e.g., as determined by bio-layer interferometry. In some embodiments, the anti-PD-L1 antibody or the fragment thereof of the present invention binds to PD-L1 (e.g., human PD-L1) with an equilibrium dissociation constant (K_{D}) of less than 3.5 nM, 3 nM, 2.5 nM, 2 nM, 1.5 nM, 1 nM, 0.9 nM, 0.8 nM, 0.7 nM, 0.6 nM, 0.5 nM, 0.4 nM, 0.3 nM, or 0.2 nM. In some embodiments, the K_{D} is above about 0.1 nM or 0.15 nM, e.g., as determined by surface plasmon resonance (SPR).

In some embodiments, the anti-PD-L1 antibody or the fragment thereof of the present invention has a lower dissociation constant (1/s) after binding to PD-L1, e.g., less than or equal to about 4×10⁻³, 3.5×10⁻³, 3×10⁻³, 2.5×10⁻³, 2×10⁻³, 1.5×10⁻³, 1.4×10⁻³, 1.3×10⁻³, 1.2×10⁻³, 1.1×10⁻³, 1×10⁻³, 9×10⁻⁴, 8×10⁻⁴, 7×10⁻⁴, 6×10⁻⁴, or 5×10⁻⁴. In some embodiments, the K_{d} is above 4×10⁻⁴ or 4.5×10⁻⁴, e.g., as determined by bio-layer interferometry or SPR.

In some embodiments, the antibody or the fragment thereof of the present invention binds to cells expressing human PD-L1, for example, with an EC₅₀ of less than or equal to about 7.5 nM, 7 nM, 6.9 nM, 6.8 nM, 6.7 nM, 6.6 nM, 6.5 nM, 6.4 nM, 6.3 nM, 6.2 nM, 6.1 nM, 6 nM, 5.9 nM, 5.8 nM, 5.7 nM, 5.6 nM, 5.5 nM, or 5.4 nM. In some embodiments, the EC₅₀ is above about 4 nM, 4.5 nM, or 5 nM. In some embodiments, the binding is determined using flow cytometry (e.g., FACS). In some embodiments, the cell expressing human PD-L1 is a CHO cell expressing human PD-L1.

In some embodiments, the antibody or the fragment thereof of the present invention blocks the relevant activity of PD-L1 (e.g., human PD-L1). In some embodiments, the blocking is superior to that of known antibodies, such as the single-domain antibody shown in SEQ ID NO: 14 in CN 107686520 A, or a heavy-chain antibody corresponding thereto. In some embodiments, the relevant activity of PD-L1 is the binding of PD-L1 to PD-1. In some embodiments, the antibody or the fragment thereof of the present invention inhibit the binding of PD-L1 to PD-1 in a mechanism of action (MOA) assay (functional biological activity detection system, e.g., from Promega). In some embodiments, the used cell is a CHO cell.

In some embodiments, the antibody or the fragment thereof of the present invention has good thermal stability. In some embodiments, the protein has a Tm of greater than or equal to about 50° C., 51° C., 52° C., 53° C., 54° C., 55° C., 56° C., 57° C., 58° C., 59° C., 60° C., or 61° C. as determined by differential scanning fluorimetry. In some embodiments, the Tm is less than or equal to about 63° C. or 62° C. In some embodiments, the antibody or the fragment thereof of the present invention has good long-term thermal stability, and, e.g., remains stable at 40° C. for at least 30 days in an accelerated stability test. In some embodiments, in the accelerated stability test, the antibody has a monomer with at least 85%, 90%, 95%, 96%, 97%, 98%, or 99% of main peak purity, e.g., at 40° C. for at least 10 days, 15 days, 20 days, 25 days, 30 days. In some embodiments, the antibody or the fragment thereof of the present invention has good solubility, which, e.g., is superior to that of known antibodies, e.g., Humira.

In some embodiments, the antibody or the fragment thereof of the present invention has better druggability.

In some embodiments, the anti-PD-L1 antibody or the fragment thereof of the present invention can induce antibody-dependent cell-mediated cytotoxicity (ADCC).

In some embodiments, the antibody of the present invention is a single-domain antibody.

In some embodiments, the antibody of the present invention is a heavy-chain antibody comprising a single-domain antibody chain of the present invention (as its heavy chain variable region). In some embodiments, the heavy-chain antibody further comprises an Fc fragment, e.g., an Fc fragment of an IgG antibody.

In some embodiments, the antibody of the present invention is a humanized, chimeric, or fully humanized antibody.

In some embodiments, the heavy chain and/or light chain of the antibody or the fragment thereof of the present invention further comprises a signal peptide sequence, such as METDTLLLWVLLLWVPGSTG (SEQ ID NO: 43).

In some embodiments, the antibody of the present invention is in a polymeric form, e.g., a heavy chain variable region in the polymeric form of a single-domain antibody and a heavy-chain antibody. In some embodiments, the antibody of the present invention is in a tetrameric form or a hexameric form.

In some embodiments, the anti-PD-L1 antibody of the present invention also comprises an antibody fragment thereof, e.g., Fab, Fab′, Fab′-SH, Fv, a single-chain antibody (e.g., scFv) or (Fab′)₂, a single-domain antibody, a diabody (dAb), and a linear antibody.

In some embodiments, the anti-PD-L1 antibody molecule is in the form of a bispecific or multispecific antibody molecule. Multispecific antibody molecule may have combinations of binding specificities for PD-L1 and any other targets.

In some embodiments, the present invention provides a nucleic acid encoding the antibody or the fragment thereof disclosed herein, a vector comprising the nucleic acid, and a host cell comprising the nucleic acid or the vector.

In some embodiments, the present invention provides a method for preparing the antibody or the fragment thereof disclosed herein.

In some embodiments, the present invention provides an immunoconjugate, a pharmaceutical composition, a kit, a combination product or an article of manufacture comprising the antibody disclosed herein.

The present invention also provides a method for mediating ADCC in a subject and a method for preventing or treating a tumor or an infection using the antibody disclosed herein. In some embodiments, the tumor is cancer.

The present invention also relates to a method for detecting PD-L1 in a sample.

The present invention also encompasses any combination of the embodiments described herein.

Any of the embodiments described herein or any combination thereof is applicable to any and all of the anti-PD-L1 antibodies or the fragments thereof, the methods, and the uses described herein.

### BRIEF DESCRIPTION OF THE DRAWINGS

FIG. 1 shows the binding level of the antibody of the present invention to cell surface antigen PD-L1.

FIG. 2 shows the inhibitory effect of the antibody of the present invention on PD-1/PD-L1 determined by MOA assay.

FIG. 3 shows the binding activity of the antibody of the present invention, AmNB1613.1, AmNB1613.12, AmNB1613.25, or AmNB1613.28, to antigen PD-L1 on CHO cells on day 0 and day 30.

FIG. 4 shows the solubility of the antibody of the present invention.

### SUMMARY

Unless defined otherwise, all technical and scientific terms used herein have the same meaning as commonly understood by those of ordinary skill in the art to which the present invention belongs. All publications, patent applications, patents, and other references mentioned herein are incorporated by reference in their entireties. In addition, the materials, methods, and examples described herein are illustrative only and are not intended to be limiting. Other features, objectives, and advantages of the present invention will be apparent from the specification and drawings, and from the appended claims.

### I. Definitions

For the purpose of explaining this specification, the following definitions will be used, and wherever appropriate, terms used in the singular form may also include the plural form, and vice versa. It should be understood that the terminology used herein is for the purpose of describing specific embodiments only, and is not intended to be limiting.

The term “about” used in combination with a numerical value is intended to encompass the numerical values in a range from a lower limit less than the specified numerical value by 5% to an upper limit greater than the specified numerical value by 5%.

As used herein, the term “and/or” refers to any one of the options or any two or more of the options.

The term “comprise” or “include” used herein, unless indicated otherwise, also encompasses the situation where the entirety consists of the described elements, integers or steps. For example, when referring to “comprise” an antibody variable region of a particular sequence, it is also intended to include an antibody variable region consisting of the particular sequence.

The term “antibody” is used herein in the broadest sense, refers to a protein comprising an antigen-binding site, and encompasses natural and artificial antibodies with various structures, including but not limited to monoclonal antibodies, polyclonal antibodies, multispecific antibodies (e.g., bispecific antibodies), single-chain antibodies, intact antibodies, and antibody fragments. Preferably, the antibody of the present invention is a single-domain antibody or a heavy-chain antibody.

“Antibody fragment” refers to a molecule different from an intact antibody, which comprises a portion of the intact antibody and binds to an antigen to which the intact antibody binds.

Examples of the antibody fragment include, but are not limited to, Fv, Fab, Fab′, Fab′-SH, and F(ab′)₂; a diabody; a linear antibody; a single-chain variable fragment (e.g., scFv); a single-domain antibody; a bivalent or bispecific antibody or a fragment thereof; a Camelidae antibody (a heavy-chain antibody); and a bispecific antibody or multispecific antibody formed from the antibody fragment.

As used herein, the term “epitope” refers to moieties of an antigen (e.g., human PD-L1) that specifically interact with an antibody molecule.

“Antibody that binds to the same or overlapping epitope” as a reference antibody refers to an antibody that blocks more than 50%, 60%, 70%, 80%, 90%, or 95% of the binding of the reference antibody to its antigen in a competition assay, or conversely, the reference antibody blocking more than 50%, 60%, 70%, 80%, 90%, or 95% of the binding of the antibody to its antigen in a competition assay.

An antibody that competes with a reference antibody to bind to its antigen refers to an antibody that blocks more than 50%, 60%, 70%, 80%, 90%, or 95% of the binding of the reference antibody to its antigen in a competition assay. Conversely, the reference antibody blocks more than 50%, 60%, 70%, 80%, 90%, or 95% of the binding of the antibody to its antigen in a competition assay. Numerous types of competitive binding assays can be used to determine whether an antibody competes with another, such as direct or indirect solid-phase radioimmunoassay (RIA), direct or indirect solid-phase enzyme immunoassay (EIA), and sandwich competition assay (see, e.g., Stahli et al., 1983, Methods in Enzymology 9:242-253). An antibody that inhibits (e.g., competitively inhibits) the binding of a reference antibody to its antigen refers to an antibody that inhibits more than 50%, 60%, 70%, 80%, 90%, or 95% of the binding of the reference antibody to its antigen. Conversely, the reference antibody inhibits more than 50%, 60%, 70%, 80%, 90%, or 95% of the binding of the antibody to its antigen. The binding of an antibody to its antigen can be measured by affinity (e.g., equilibrium dissociation constant). Methods for determining affinity are known in the art, e.g., SPR or bio-layer interferometry technology.

An antibody that shows the same or similar binding affinity and/or specificity as a reference antibody refers to an antibody that is capable of having at least more than 50%, 60%, 70%, 80%, 90%, or 95% of the binding affinity and/or specificity of the reference antibody. This can be determined by any method known in the art for determining binding affinity and/or specificity. “Complementarity determining region” or “CDR region” or “CDR” is a region in an antibody variable domain that is highly variable in sequence and forms a structurally defined loop (“hypervariable loop”) and/or comprises antigen-contacting residues (“antigen contact site”). CDRs are primarily responsible for binding to antigen epitopes. The CDRs of the heavy and light chains are generally referred to as CDR1, CDR2, and CDR3, and are numbered sequentially from the N-terminus. The CDRs located in the heavy chain variable domain of the antibody are referred to as HCDR1, HCDR2 and HCDR3, whereas the CDRs located in the light chain variable domain of the antibody are referred to as LCDR1, LCDR2 and LCDR3. In a given amino acid sequence of a light chain variable region or a heavy chain variable region, the exact amino acid sequence boundary of each CDR can be determined using any one or a combination of many well-known antibody CDR assignment systems including, e.g., Chothia based on the three-dimensional structure of antibodies and the topology of the CDR loops (Chothia et al. (1989) Nature 342:877-883; Al-Lazikani et al., Standard conformations for the canonical structures of immunoglobulins, Journal of Molecular Biology, 273:927-948 (1997)), Kabat based on antibody sequence variability (Kabat et al., Sequences of Proteins of Immunological Interest, 4^{th} ed, U.S. Department of Health and Human Services, National Institutes of Health (1987)), AbM (University of Bath), Contact (University College London), International ImMunoGeneTics database (IMGT) (imgt.cines.fr/ on the World Wide Web), and North CDR definition based on the affinity propagation clustering using a large number of crystal structures. Unless otherwise stated, the term “CDR” or “CDR sequence” used herein encompasses CDR sequences determined by any one of the schemes above.

CDRs can also be determined based on having the same Chothia numbering positions as a reference CDR sequence (e.g., any one of the exemplary CDRs of the present invention). In one embodiment, the CDR1, CDR2 and CDR3 of the antibody of the present invention comprise amino acid residues at positions 26-35, amino acid residues at positions 50-58 and amino acid residues at positions 95-102, respectively, according to Chothia numbering positions based on the AbM rule.

Unless otherwise stated, residue positions of antibody variable regions and CDRs (including heavy chain variable region residues) are numbered according to the Chothia numbering system. In one embodiment, the boundaries of the CDRs of the antibody of the present invention are determined as per the AbM scheme.

Antibodies with different specificities (i.e., different binding sites for different antigens) have different CDRs. However, although CDRs differ from antibody to antibody, only a limited number of amino acid positions within the CDRs are directly involved in antigen binding. The smallest overlapping region can be determined using at least two of the Kabat, Chothia, AbM, and Contact schemes, thereby providing a “minimal binding unit” for antigen binding. The minimal binding unit may be a sub-portion of the CDR. As will be appreciated by those skilled in the art, residues in remaining portions of the CDR sequences can be determined by the structure and protein folding of the antibody. Thus, variants of any CDR presented herein are also considered. For example, in a variant of one CDR, the amino acid residue of the minimal binding unit may remain unchanged, while the remaining CDR residues defined by the Kabat or Chothia may be conservatively substituted.

The term “single-domain antibody (V_{H}H)” generally refers to an antibody comprising only one heavy chain variable region, and having an activity of binding to an antigen, i.e., an antibody comprising only one chain of FR4-VCDR3-FR3-VCDR2-FR2-VCDR1-FR1 from C-terminus to N-terminus, which may be produced naturally in camels or by genetic engineering techniques. A single-domain antibody is the minimal unit known to bind target antigens.

As used herein, the term “heavy-chain antibody (hcAb)” refers to an antibody without a light chain, which may comprise VH-CH2-CH3 or VH-CH1-CH2-CH3 from N-terminus to C-terminus, and a homodimer thereof, such as a heavy-chain antibody dimer without light chains. The heavy-chain antibody of the present invention may comprise a VH from a standard antibody or a VH from a single-domain antibody. For example, the VH in the heavy-chain antibody of the present invention may be a single-domain antibody.

As used herein, the term “multispecific antibody” refers to an antibody having at least two antigen-binding sites, each of which binds to a different epitope of the same antigen or a different epitope of a different antigen. A multispecific antibody is an antibody having binding specificities for at least two different epitopes. In one embodiment, provided herein is a bispecific antibody having binding specificities for a first antigen and a second antigen.

The term “effector function” refers to bioactivities attributed to an immunoglobulin Fc region that vary with immunoglobulin isotype. Examples of immunoglobulin effector functions include: C1q binding and complement-dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake in antigen-presenting cells, down regulation of cell surface receptors (such as B-cell receptors), and B-cell activation.

The term “chimeric antibody” is an antibody molecule in which: (a) a constant region or a portion thereof is modified, substituted, or exchanged such that antigen-binding sites are linked to constant regions of different or modified classes, effector functions and/or species, or disparate molecules imparting new properties (e.g., enzymes, toxins, hormones, growth factors, and drugs) to chimeric antibodies, etc.; or (b) a constant region or a portion thereof is modified, substituted, or exchanged by variable regions with different or modified antigen binding specificities. For example, a mouse antibody can be modified by substituting its constant region for a constant region from a human immunoglobulin. Due to the substitution of a human constant region, the chimeric antibody can retain its specificity for recognizing antigens, while having reduced antigenicity in humans as compared to the original mouse antibody.

“Humanized” antibody refers to a chimeric antibody comprising amino acid residues from non-human CDRs and amino acid residues from human FRs. In some embodiments, a humanized antibody will comprise at least one, or generally two of substantially all variable domains in which all or substantially all CDRs (e.g., CDRs) correspond to those of a non-human antibody, and all or substantially all FRs correspond to those of a human antibody. A humanized antibody may optionally comprise at least a portion of an antibody constant region derived from a human antibody. The “humanized form” of an antibody (such as a non-human antibody) refers to an antibody that has been humanized.

“Human antibody” refers to an antibody having an amino acid sequence which corresponds to the amino acid sequence of an antibody generated by a human or human cell or derived from a non-human source that utilizes human antibody libraries or other human antibody encoding sequences. This definition of a human antibody explicitly excludes humanized antibodies comprising non-human antigen-binding residues.

The term “Fc region” is used herein to define a C-terminus region of an immunoglobulin heavy chain, which comprises at least one portion of a constant region. The “Fc region” includes Fc regions of native sequences and variant Fc regions. In certain embodiments, a human IgG heavy chain Fc region extends from Cys226 or Pro230 to the carbonyl end of the heavy chain. However, the C-terminal lysine (Lys447) of the Fc region may or may not be present. Unless otherwise stated, the numbering of amino acid residues in the Fc region or constant region is based on an EU numbering system, which is also called EU index as described in Kabat et al., Sequences of Proteins of Immunological Interest, 5^{th} ed. Public Health Service, National Institutes of Health, Bethesda, Md., 1991.

The term “variable region” or “variable domain” refers to a domain of a heavy chain or light chain of an antibody involved in the binding of the antibody to an antigen. Variable domains of heavy chains and light chains of native antibodies often have similar structures, wherein each domain contains four conserved framework regions (FR) and three complementarity determining regions (CDR). (See, for example, Kindt et al., Kuby Immunology, 6^{th} ed., W.H. Freeman and Co., p 91 (2007)). A single VH or VL domain may be sufficient to provide antigen-binding specificity.

As used herein, the term “binding” or “specific binding” means that the binding effect is selective for antigens and can be distinguished from unwanted or non-specific interactions. The ability of an antibody to bind to a particular antigen can be determined by an enzyme-linked immunosorbent assay (ELISA), SPR or bio-layer interferometry or a conventional binding assay known in the art.

The term “cytokine” is a generic term for proteins that are released by a cell population and act as intercellular mediators on another cell. As used herein, the term “cytokine” includes proteins from natural sources or from recombinant cell cultures and biologically active equivalents of native sequence cytokines, including small molecule entities produced by artificial synthesis, and pharmaceutically acceptable derivatives and salts thereof.

“Immunoconjugate” is an antibody conjugated to one or more other substances, including but not limited to cytotoxic agents or labels.

The terms “programmed cell death 1 ligand 1”, “PD-L1”, “programmed death ligand 1”, “cluster of differentiation 274”, “CD274”, or “B7 homolog 1” as used herein refer to any natural PD-L1 from any vertebrate source including mammals such as primates (e.g., humans) and rodents (e.g., mice and rats). The terms encompass “full length”, unprocessed PD-L1, and PD-L1 of any form that results from processing in the cell. PD-L1 may present as a transmembrane protein or as a soluble protein. The terms also encompass variants of naturally occurring PD-L1, such as splicing variants or allelic variants. The basic structure of PD-L1 includes four domains: an extracellular Ig-like V-type domain and an Ig-like C2-type domain, a transmembrane domain and a cytoplasmic domain. Additional information about the human PD-L1 gene including genomic DNA sequences can be found under NCBI Gene ID No. 29126. Additional information about the human PD-L1 gene including genomic DNA sequences can be found under NCBI Gene ID No. 60533. The amino acid sequence of an exemplary full-length human PD-L1 protein can be found, e.g., under NCBI accession number NP_001254653 or UniProt accession number Q9NZQ7, and the sequence of an exemplary full-length mouse PD-L1 protein can be found, e.g., under NCBI accession number NP_068693 or Uniprot accession number Q9EP73.

The terms “anti-PD-L1 antibody”, “anti-PD-L1”, “PD-L1 antibody” or “PD-L1-binding antibody” as used herein refer to antibodies capable of binding PD-L1 protein with sufficient affinity, or fragments thereof. In one embodiment, the extent to which the anti-PD-L1 antibody binds to a non-PD-L1 protein is less than about 10%, about 20%, about 30%, about 40%, about 50%, about 60%, about 70%, about 80%, or about 90%, or more of the extent to which the antibody binds to a PD-L1 protein, as measured, e.g., by radioimmunoassay (RIA) or bio-optical interference assay or MSD assay or SPR or bio-layer interferometry.

The term “inhibitor” or “antagonist” includes substances that reduce certain parameters (e.g., activity) of a given molecule (e.g., an immune checkpoint molecule). For example, the “inhibitor” or “antagonist” includes substances that inhibit the activity (e.g., PD-1 activity) of a given molecule by at least 5%, 10%, 20%, 30%, 40%, or more. Therefore, the inhibitory effect needs not be 100%. The term “activator” includes substances that increase certain parameters (e.g., activity) of a given molecule (e.g., a co-stimulatory molecule). For example, the “activator” includes substances that increase the activity (e.g., OX40 activity) of a given molecule by at least 5%, 10%, 20%, 30%, 40%, or more. Therefore, the activation effect need not be 100%.

“Functional Fc region” possesses the “effector functions” of Fc regions of native sequences. Exemplary “effector functions” include C1q binding, CDC, Fc receptor binding, ADCC, phagocytosis, cell surface receptor (e.g., B cell receptor, or BCR) down-regulation, and the like. Such effector functions generally require that the Fc region is associated with a binding domain (e.g., an antibody variable domain) and can be assessed using a variety of assays, such as those disclosed herein.

“Effector function” refers to biological activities which can be attributed to the antibody Fc region and vary with the antibody isotype. Examples of antibody effector functions include: C1q binding and complement-dependent cytotoxicity (CDC), Fc receptor binding, antibody-dependent cell-mediated cytotoxicity (ADCC), phagocytosis, cell surface receptors (such as B cell receptors) down-regulation, and B cell activation.

“Human effector cell” refers to a leukocyte that expresses one or more FcRs and executes effector functions. In certain embodiments, the cell expresses at least Fc and executes effector function of ADCC. Examples of human leukocytes mediating ADCC include peripheral blood mononuclear cells (PBMC), natural killer (NK) cells, monocytes, cytotoxic T cells, and neutrophils. Effector cells can be isolated from their natural sources, such as blood.

The term “effective amount” refers to an amount or dosage of the antibody, fragment, conjugate or composition disclosed herein which generates expected effects in a patient in need of treatment or prevention after administered to the patient in a single or multiple doses. The effective amount can be easily determined by an attending physician as a person skilled in the art by considering a variety of factors as follows: species such as mammals; its size, age, and general health condition; the specific disease involved; the extent or severity of the disease; response in an individual patient; specific antibody administered; route of administration; bioavailability characteristics of the administered preparation; selected dosage regimen; and uses of any concomitant therapy.

The “therapeutically effective amount” refers to an amount effective to achieve a desired therapeutic result at a necessary dosage for a necessary period of time. The therapeutically effective amount of an antibody or antibody fragment, or conjugate or composition thereof may vary depending on a variety of factors such as disease state, age, sex, and weight of an individual, and the ability of the antibody or antibody portion to elicit a desired response in an individual. The therapeutically effective amount is also such an amount that any toxic or undesired effect of the antibody or the fragment thereof, or conjugate or composition thereof is inferior to the therapeutically beneficial effect. “Therapeutically effective amount” preferably inhibits a measurable parameter (e.g., tumor growth rate and tumor volume) by at least about 20%, more preferably at least about 40%, even more preferably at least about 50%, 60%, or 70%, and still more preferably at least about 80% or 90%, relative to untreated subjects. The ability of a compound to inhibit a measurable parameter (e.g., cancer) can be evaluated in an animal model system that predicts efficacy in human tumors.

The “prophylactically effective amount” refers to an amount effective to achieve a desired prophylactic result at a necessary dosage for a necessary period of time. Generally, since a prophylactic dose is administered in a subject before or at an earlier stage of a disease, a prophylactically effective amount will be less than a therapeutically effective amount.

The term “individual” or “subject” can be used interchangeably and includes mammals. The mammals include, but are not limited to, domesticated animals (e.g., cows, sheep, cats, dogs, and horses), primates (e.g., human and non-human primates such as monkeys), rabbits and rodents (e.g., mice and rats). In particular, the individual or subject is a human.

The terms “tumor” and “cancer” are used interchangeably herein and encompass solid and liquid tumors.

The terms “cancer” and “cancerous” refer to or describe a physiological disease in mammals that is typically characterized by unregulated cell growth.

The term “tumor” refers to all neoplastic cell growth and proliferation regardless of whether malignant or benign, and all pre-cancerous and cancerous cells and tissues. The terms “cancer”, “cancerous” and “tumor” are not mutually exclusive when referred to herein.

The term “infectious disease” refers to a disease caused by a pathogen.

The term “label” used herein refers to a compound or composition which is directly or indirectly conjugated or fused to an agent, such as a polynucleotide probe or an antibody, and facilitates the detection of the agent to which it is conjugated or fused. The label itself can be detectable (e.g., a radioisotope label or a fluorescent label) or can catalyze a chemical change to a detectable substrate compound or composition in the case of enzymatic labeling. The term is intended to encompass direct labeling of a probe or an antibody by coupling (i.e., physical linking) a detectable substance to the probe or the antibody and indirect labeling of a probe or an antibody by reacting with another reagent which is directly labeled. Examples of indirect labeling include detection of a primary antibody using a fluorescently labeled secondary antibody, and end labeling of a biotinylated DNA probe such that it can be detected with a fluorescently labeled streptavidin.

An “isolated” antibody is an antibody which has been separated from components of its natural environment. In some embodiments, the antibody is purified to a purity greater than 95% or 99% as determined by, e.g., electrophoresis (e.g., SDS-PAGE, isoelectric focusing (IEF) and capillary electrophoresis) or chromatography (e.g., ion exchange or reverse-phase HPLC). For a review of methods for assessing antibody purity, see, for example, Flatman et al., J. Chromatogr., B848:79-87 (2007).

An “isolated” nucleic acid is a nucleic acid molecule which has been separated from components of its natural environment. The isolated nucleic acid includes a nucleic acid molecule contained in a cell that typically comprises the nucleic acid molecule, but the nucleic acid molecule exists extrachromosomally or at a chromosomal location that is different from its natural chromosomal location. “An isolated nucleic acid encoding an anti-PD-L1 antibody or a fragment thereof” refers to one or more nucleic acid molecules encoding chains of an antibody or a fragment thereof, including such nucleic acid molecules in a single vector or separate vectors, and such nucleic acid molecules present at one or more locations in a host cell.

The calculation of sequence identity between sequences is performed as follows.

To determine the percent identity of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison). In one preferred embodiment, for comparison purposes, the length of the aligned reference sequence is at least 30%, preferably at least 40%, more preferably at least 50% or 60%, and even more preferably at least 70%, 80%, 90%, or 100% of the length of the reference sequence. Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

A mathematical algorithm can be used to compare the sequences and calculate percent identity between two sequences. In one preferred embodiment, the percent identity between two amino acid sequences is determined with the Needlema and Wunsch algorithm ((1970) J. Mol. Biol., 48:444-453; available at http://www.gcg.com) which has been integrated into the GAP program of the GCG software package, using the Blossom 62 matrix or PAM250 matrix and gap weights of 16, 14, 12, 10, 8, 6, or 4 and length weights of 1, 2, 3, 4, 5, or 6. In another preferred embodiment, the percent identity between two nucleotide acid sequences is determined with the GAP program (available at http://www.gcg.com) of the GCG software package, using the NWSgapdna.CMP matrix and gap weights of 40, 50, 60, 70, or 80 and length weights of 1, 2, 3, 4, 5, or 6. A particularly preferred parameter set (and one that should be used unless otherwise stated) is a Blossom 62 scoring matrix with a gap penalty of 12, a gap extension penalty of 4, and a frameshift gap penalty of 5.

The percent identity between two amino acid sequences or nucleotide sequences can also be determined with PAM120 weighted remainder table, gap length penalty of 12 and gap penalty of 4, using the E. Meyers and W. Miller algorithms which have been incorporated into the ALIGN program (version 2.0) ((1989) CABIOS, 4:11-17).

Additionally or alternatively, the nucleic acid sequences and protein sequences described herein can be further used as “query sequences” to perform searches against public databases to, e.g., identify other family member sequences or related sequences.

As used herein, the term “hybridization under stringency conditions (such as low stringency, medium stringency, high stringency or extreme stringency)” describes hybridization and washing conditions. Instructions for performing hybridization reactions can be found in Current Protocols in Molecular Biology, John Wiley & Sons, N.Y. (1989), 6.3.1-6.3.6, which is incorporated by reference. Aqueous and non-aqueous methods are described in the references and either method can be used. In some embodiments, the specific hybridization conditions mentioned herein are as followed: 1) low stringency hybridization conditions are in 6× sodium chloride/sodium citrate (SSC) at about 45° C., followed by two washes in 0.2×SSC, 0.1% SDS at least at 50° C. (for low stringency conditions, the temperature of the washes can be increased to 55° C.); 2) medium stringency hybridization conditions are in 6×SSC at about 45° C., followed by one or more washes in 0.2×SSC, 0.1% SDS at about 60° C.; 3) high stringency hybridization conditions are in 6×SSC at about 45° C., followed by one or more washes in 0.2×SSC, 0.1% SDS at 65° C.; and preferably 4) extreme stringency hybridization conditions are in 0.5 M sodium phosphate, 7% SDS at 65° C., followed by one or more washes in 0.2×SSC, 0.1% SDS at 65° C. Extreme stringency condition (4) is a preferred condition and the one that should be used unless otherwise stated.

The term “pharmaceutical composition” refers to such a composition that exists in a form allowing effective biological activity of the active ingredient contained therein, and does not contain additional ingredients having unacceptable toxicity to a subject to which the composition is administered.

The term “pharmaceutical excipient” refers to diluents, adjuvants (e.g., Freund's adjuvants (complete and incomplete)), carriers, excipients or stabilizers, etc., which are co-administered with active substance.

As used herein, “treatment” (or “treat” or “treating”) refers to slowing, interrupting, arresting, alleviating, stopping, reducing, or reversing the progression or severity of an existing symptom, disorder, condition, or disease. Desired therapeutic effects include, but are not limited to, preventing the occurrence or recurrence of diseases, alleviating symptoms, reducing any direct or indirect pathological outcomes of diseases, preventing metastasis, delaying disease progression, improving or alleviating conditions, and improving prognosis. In some embodiments, the antibody molecule of the present invention is used to delay the progression of a disease or to slow the progression of a disease.

As used herein, “prevention” (or “prevent” or “preventing”) includes the inhibition of the onset or progression of a disease or disorder or a symptom of a specific disease or disorder. In some embodiments, subjects with family history of cancer are candidates for preventive regimens.

Generally, in the context of cancer, the term “prevention” refers to the administration of a drug prior to the onset of signs or symptoms of a cancer, particularly in subjects at risk of cancer.

The term “therapeutic agent” described herein encompasses any substance effective in preventing or treating tumors (e.g., cancer) and infections, including chemotherapeutic agents, cytotoxic agents, vaccines, other antibodies, anti-infective active agents, small molecule drugs, or immunomodulatory agents.

“Chemotherapeutic agents” include chemical compounds useful in treatment of cancer.

The term “immunomodulatory agent” used herein refers to a natural or synthetic active agent or drug that suppresses or modulates an immune response. The immune response may be a humoral response or a cellular response.

The term “small molecule drug” refers to a low molecular weight organic compound capable of regulating biological processes.

As used herein, the term “cytotoxic agent” refers to a substance that inhibits or prevents cell function and/or causes cell death or destruction.

The term “anti-infective agent” includes any molecule that specifically inhibits or eliminates the growth of microorganisms at the administration concentration and interval of administration. In one specific aspect, the anti-infective agent is non-toxic to the host at the administration concentration and interval of administration.

The term “combination product” refers to a kit with a fixed combination, a non-fixed combination, or a part for combined administration in the form of a dose unit, wherein two or more therapeutic agents can be independently administered simultaneously or separately administered at certain intervals of time, especially when these intervals allow combination partners to exhibit collaboration, such as synergistic effect. The term “fixed combination” means that the antibody disclosed herein and a combination partner (e.g., other therapeutic agents) are simultaneously administered to a patient in the form of a single entity or dose. The term “non-fixed combination” means that the antibody disclosed herein and a combination partner (e.g., other therapeutic agents) are simultaneously, concurrently, or sequentially administered to a patient as separate entities, without specific time limitation, wherein such administration provides therapeutically effective levels of the two therapeutic agents in the patient. The latter is also applicable to a cocktail therapy, e.g., administration of three or more therapeutic agents. In one preferred embodiment, the drug combination is a non-fixed combination.

The term “combination therapy” or “combined therapy” means that two or more therapeutic agents are administered to treat a cancer or an infection as described herein. Such administration includes co-administration of these therapeutic agents in a substantially simultaneous manner, for example, in a single capsule with a fixed proportion of active ingredients. Alternatively, such administration includes co-administration or separate administration or sequential administration of each active ingredient in a variety of or separate containers (such as tablets, capsules, powder and liquid). The powder and/or liquid can be reconstituted or diluted to a desired dosage before administration. In some embodiments, the administration also includes using each type of therapeutic agents in a sequential manner at approximately the same time or at different times. In any case, the therapeutic regimen will provide the beneficial effect of the drug combination in the treatment of disorders or symptoms described herein.

The term “vector” used herein refers to a nucleic acid molecule capable of proliferating another nucleic acid to which it is linked. The term includes vectors that serve as self-replicating nucleic acid structures as well as vectors binding to the genome of a host cell into which they have been introduced. Some vectors are capable of directing the expression of a nucleic acid to which they are operatively linked. Such vectors are called “expression vectors” herein.

The term “host cell” refers to a cell into which an exogenous polynucleotide has been introduced, including progeny of such cells. Host cells include “transformants” and “transformed cells”, which include primary transformed cells and progeny derived therefrom, regardless of the number of passages. A generation may not be completely identical in nucleic acid content to the parent cell, but may contain mutations. Mutant generations having the same function or bioactivity that are screened or selected from the initially transformed cells are included herein. Host cells are any type of cell systems that can be used to produce the antibody molecule of the present invention, including eukaryotic cells, e.g., mammalian cells, insect cells, and yeast cells; and prokaryotic cells, e.g., E. coli cells. Host cells include cultivated cells, as well as cells within a transgenic animal, a transgenic plant, or a cultivated plant tissue or an animal tissue.

“Subject/patient sample” refers to a collection of cells or fluids obtained from a patient or a subject. The source of tissue or cell samples can be solid tissues, e.g., from fresh, frozen and/or preserved organ or tissue samples or biopsy samples or puncture samples; blood or any blood component; body fluids such as cerebrospinal fluids, amniotic fluids, peritoneal fluids, or interstitial fluids; and cells from a subject at any time during pregnancy or development. Tissue samples may comprise compounds which are naturally not mixed with tissues, such as preservatives, anticoagulants, buffers, fixatives, nutrients, antibiotics, and the like. Examples of tumor samples include but are not limited to tumor biopsies, fine needle aspirates, bronchial lavage fluids, pleural fluids, sputa, urine, surgical specimens, circulating tumor cells, serum, plasma, circulating plasma proteins, ascites, primary cell cultures or cell lines derived from tumors or exhibiting tumor-like properties, and preserved tumor samples such as formalin-fixed, paraffin-embedded tumor samples or frozen tumors samples.

The term “package insert” is used to refer to the instructions generally contained in the commercial package of therapeutic products, which contain information about indications, usage, dosage, administration, combination therapies, contraindications and/or warnings related to the application of such therapeutic products.

### II. Antibody of the Present Invention

In some embodiments, the antibody or the fragment thereof of the present invention binds to PD-L1. In some embodiments, the antibody or the fragment thereof of the present invention binds to mammalian PD-L1, such as human PD-L1. For example, the antibody molecule specifically binds to an epitope (e.g., a linear or conformational epitope) of PD-L1. In some embodiments, the antibody molecule binds to one or more extracellular domains of PD-L1.

In some embodiments, the anti-PD-L1 antibody or the antigen-binding fragment thereof of the present invention has one or more of the following characteristics:

(i) showing the same or similar binding affinity and/or specificity for PD-L1 as the antibody of the present invention;

(ii) inhibiting (e.g., competitively inhibiting) the binding of the antibody of the present invention to PD-L1;

(iii) binding to the same or overlapping epitope as the antibody of the present invention;

(iv) competing with the antibody of the present invention for binding to PD-L1;

(v) having one or more biological properties of the antibody of the present invention.

In some embodiments, the anti-PD-L1 antibody or the antigen-binding fragment thereof of the present invention comprises:

(i) three complementarity determining regions (CDRs) in a VH set forth in any one of SEQ ID NOs: 14 to 18, or

(ii) relative to the sequence of (i), a sequence comprising a total of at least one and no more than 5, 4, 3, 2, or 1 amino acid alteration (preferably amino acid replacement, and more preferably conservative replacement) in the three CDRs.

In some embodiments, the anti-PD-L1 antibody or the antigen-binding fragment thereof of the present invention comprises or consists of a heavy chain variable region comprising:

(i) three complementarity determining regions (CDRs) in a VH set forth in any one of SEQ ID NOs: 14 to 18, or

(ii) relative to the sequence of (i), a sequence comprising a total of at least one and no more than 5, 4, 3, 2, or 1 amino acid alteration (preferably amino acid replacement, and more preferably conservative replacement) in the three CDRs.

In some embodiments, the anti-PD-L1 antibody or the antigen-binding fragment thereof of the present invention comprises:

complementary determining regions (CDRs) HCDR1, HCDR2, and HCDR3, wherein the HCDR1 comprises or consists of an amino acid sequence selected from SEQ ID NOs: 1, 2, 3, and 44, or the HCDR1 comprises an amino acid sequence having one, two, or three alternations (preferably amino acid substitutions, more preferably conservative replacements) compared to an amino acid sequence selected from SEQ ID NOs: 1, 2, 3, and 44; the HCDR2 comprises or consists of an amino acid sequence set forth in SEQ ID NO: 4, or the HCDR2 comprises an amino acid sequence having one, two, or three alternations (preferably amino acid substitutions, more preferably conservative replacements) compared to an amino acid sequence set forth in SEQ ID NO: 4; the HCDR3 comprises or consists of an amino acid sequence selected from SEQ ID NOs: 5, 6, 7, 8, and 45, or the HCDR3 comprises an amino acid sequence having one, two, or three alternations (preferably amino acid substitutions, more preferably conservative replacements) compared to an amino acid sequence selected from SEQ ID NOs: 5, 6, 7, 8, and 45.

In some embodiments, the anti-PD-L1 antibody or the antigen-binding fragment thereof of the present invention comprises or consists of a heavy chain variable region comprising: complementary determining regions (CDRs) HCDR1, HCDR2, and HCDR3, wherein the HCDR1 comprises or consists of an amino acid sequence selected from SEQ ID NOs: 1, 2, 3, and 44, or the HCDR1 comprises an amino acid sequence having one, two, or three alternations (preferably amino acid substitutions, more preferably conservative replacements) compared to an amino acid sequence selected from SEQ ID NOs: 1, 2, 3, and 44; the HCDR2 comprises or consists of an amino acid sequence set forth in SEQ ID NO: 4, or the HCDR2 comprises an amino acid sequence having one, two, or three alternations (preferably amino acid substitutions, more preferably conservative replacements) compared to an amino acid sequence set forth in SEQ ID NO: 4; the HCDR3 comprises or consists of an amino acid sequence selected from SEQ ID NOs: 5, 6, 7, 8, and 45, or the HCDR3 comprises an amino acid sequence having one, two, or three alternations (preferably amino acid substitutions, more preferably conservative replacements) compared to an amino acid sequence selected from SEQ ID NOs: 5, 6, 7, 8, and 45.

In some embodiments, the anti-PD-L1 antibody or the antigen-binding fragment thereof of the present invention comprises or consists of a heavy chain variable region comprising: complementarity determining regions (CDRs) HCDR1, HCDR2 and HCDR3, wherein the HCDR3 comprises or consists of an amino acid sequence selected from SEQ ID NOs: 5, 6, 7, 8, and 45, or the HCDR3 comprises an amino acid sequence having one, two, or three alterations (preferably amino acid substitutions, more preferably conservative replacements) compared to an amino acid sequence selected from SEQ ID NOs: 5, 6, 7, 8, and 45.

In one embodiment, the anti-PD-L1 antibody or the antigen-binding fragment thereof of the present invention comprises complementarity determining regions (CDRs) HCDR1, HCDR2, and HCDR3, wherein

(i) the HCDR1 comprises or consists of an amino acid sequence set forth in SEQ ID NO: 2, the HCDR2 comprises or consists of an amino acid sequence set forth in SEQ ID NO: 4, and the HCDR3 comprises or consists of an amino acid sequence set forth in SEQ ID NO: 5;

(ii) the HCDR1 comprises or consists of an amino acid sequence set forth in SEQ ID NO: 3, the HCDR2 comprises or consists of an amino acid sequence set forth in SEQ ID NO: 4, and the HCDR3 comprises or consists of an amino acid sequence set forth in SEQ ID NO: 5;

(iii) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 6;

(iv) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 7;

(v) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 8; or

(vi) the HCDR1 comprises or consists of an amino acid sequence set forth in SEQ ID NO: 44, the HCDR2 comprises or consists of an amino acid sequence set forth in SEQ ID NO: 4, and the HCDR3 comprises or consists of an amino acid sequence set forth in SEQ ID NO: 45.

In one embodiment, the anti-PD-L1 antibody or the antigen-binding fragment thereof of the present invention comprises or consists of a heavy chain variable region comprising complementarity determining regions (CDRs) HCDR1, HCDR2 and HCDR3, wherein

(i) the HCDR1 comprises or consists of an amino acid sequence set forth in SEQ ID NO: 2, the HCDR2 comprises or consists of an amino acid sequence set forth in SEQ ID NO: 4, and the HCDR3 comprises or consists of an amino acid sequence set forth in SEQ ID NO: 5;

(ii) the HCDR1 comprises or consists of an amino acid sequence set forth in SEQ ID NO: 3, the HCDR2 comprises or consists of an amino acid sequence set forth in SEQ ID NO: 4, and the HCDR3 comprises or consists of an amino acid sequence set forth in SEQ ID NO: 5;

(iii) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 6;

(iv) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 7;

(v) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 8; or

(vi) the HCDR1 comprises or consists of an amino acid sequence set forth in SEQ ID NO: 44, the HCDR2 comprises or consists of an amino acid sequence set forth in SEQ ID NO: 4, and the HCDR3 comprises or consists of an amino acid sequence set forth in SEQ ID NO: 45.

In some embodiments, the anti-PD-L1 antibody or the antigen-binding fragment thereof of the present invention comprises or consists of a heavy chain variable region, wherein the heavy chain variable region:

(i) comprises or consists of an amino acid sequence having at least 90%, 91%, 92%, 93%, 94%, 95%, 96%, 97%, 98% or 99% identity to an amino acid sequence selected from any one of SEQ ID NOs: 14 to 18;

(ii) comprises or consists of an amino acid sequence selected from any one of SEQ ID NOs: 14 to 18; or

(iii) comprises an amino acid sequence having 1 or more (preferably not more than 10, more preferably not more than 5, 4, 3, 2 or 1) amino acid alterations (preferably amino acid substitutions, more preferably amino acid conservative replacements) as compared to an amino acid sequence selected from any one of SEQ ID NOs: 14 to 18, wherein preferably, the amino acid alterations do not occur in the CDRs.

In some embodiments, the anti-PD-L1 antibody or the antigen-binding fragment thereof of the present invention further comprises an Fc region, preferably the Fc region being linked to the C terminus of the heavy chain variable region. In some embodiments, the antibody of the present invention further comprises a constant region CH1 between the heavy chain variable region and the Fc region. In some embodiments, the Fc region is from an IgG, e.g., IgG1, IgG2, IgG3, or IgG4. In some embodiments, the Fc region is from IgG1. In some embodiments, the Fc region is from human IgG1 LALA. In some embodiments, the Fc region:

(i) comprises or consists of an amino acid sequence having at least 85%, 90%, 91%, 92%, 93%, 94%, 95%, 96%, 97%, 98%, or 99% identity to an amino acid sequence set forth in SEQ ID NO: 25;

(ii) comprises or consists of an amino acid sequence set forth in SEQ ID NO: 25; or

(iii) comprises an amino acid sequence having 1 or more (preferably not more than 10, more preferably not more than 5, 4, 3, 2 or 1) amino acid alterations (preferably amino acid substitutions, more preferably amino acid conservative replacements) as compared to an amino acid sequence set forth in SEQ ID NO: 25.

In some embodiments, the anti-PD-L1 antibody or the antigen-binding fragment thereof of the present invention comprises or consists of a heavy chain, wherein the heavy chain:

(i) comprises or consists of an amino acid sequence having at least 85%, 90%, 91%, 92%, 93%, 94%, 95%, 96%, 97%, 98% or 99% identity to an amino acid sequence selected from any one of SEQ ID NOs: 20 to 24;

(ii) comprises or consists of an amino acid sequence selected from any one of SEQ ID NOs: 20 to 24; or

(iii) comprises an amino acid sequence having 1 or more (preferably not more than 10, more preferably not more than 5, 4, 3, 2 or 1) amino acid alterations (preferably amino acid substitutions, more preferably amino acid conservative replacements) as compared to an amino acid sequence selected from any one of SEQ ID NOs: 20 to 24, wherein preferably, the amino acid alterations do not occur in the CDRs.

In some embodiments, the anti-PD-L1 antibody of the present invention is a single-domain antibody comprising or consisting of the heavy chain variable region VH as defined herein.

In some embodiments, the anti-PD-L1 antibody of the present invention is a heavy-chain antibody, e.g., comprising the VH region, the Fc region, and optionally the CH1 region as defined herein.

In some embodiments, the heavy chain and/or light chain of the anti-PD-L1 antibody or the fragment thereof of the present invention further comprises a signal peptide sequence, such as METDTLLLWVLLLWVPGSTG (SEQ ID NO: 43).

In one embodiment of the present invention, the amino acid alteration described herein includes amino acid replacement, insertion or deletion. Preferably, the amino acid alteration described herein is an amino acid replacement, preferably a conservative replacement.

In a preferred embodiment, the amino acid alteration described herein occurs in a region outside the CDR (e.g., in FR). More preferably, the amino acid alteration described herein occurs in a region outside the heavy chain variable region and/or outside the light chain variable region. In some embodiments, the replacement is a conservative replacement. A conservative replacement refers to the replacement of an amino acid by another amino acid of the same class, e.g., the replacement of an acidic amino acid by another acidic amino acid, the replacement of a basic amino acid by another basic amino acid, or the replacement of a neutral amino acid by another neutral amino acid. Exemplary replacements are shown in Table A below:

TABLE A

| Original | | Preferred |
|---|---|---|
| residues | Exemplary replacement | replacement |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu, Val; Met; Ala; Phe; norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; norleucine | Leu |

In certain embodiments, the antibody provided herein is altered to increase or decrease the extent to which the antibody is glycosylated. Addition or deletion of glycosylation sites to an antibody can be conveniently achieved by altering the amino acid sequence such that one or more glycosylation sites are created or removed. When the antibody comprises an Fc region, carbohydrate attached thereto can be altered. In some applications, removing undesired modifications to glycosylation sites can be useful, for example, removing fucose modules to enhance antibody-dependent cellular cytotoxicity (ADCC) (see Shield et al., (2002) JBC277:26733). In other applications, galactosidylation modification can be carried out to modify complement-dependent cytotoxicity (CDC). In certain embodiments, one or more amino acid modifications can be introduced into the Fc region of the antibody provided herein, thereby producing an Fc region variant such that, for example, the efficacy of the antibody in treating cancer or a cell proliferative disease is enhanced. The Fc region variant may comprise a human Fc region sequence (such as human IgG1, IgG2, IgG3, or IgG4 Fc region) comprising an amino acid modification (such as replacement) at one or more amino acid positions.

In one embodiment, the number of cysteine residues of an antibody can be altered to modify antibody properties. For example, the hinge region of CH1 is modified to change (e.g., increase or decrease) the number of cysteine residues in the hinge region. This method is further described in U.S. Pat. No. 5,677,425. The number of cysteine residues in the hinge region of CH1 can be changed to, e.g., facilitate assembly of the light and heavy chains, or increase or decrease the stability of the antibody.

Optionally, the antibody of the present invention comprises post-translational modifications to the antibody chain. Exemplary post-translational modifications include disulfide bond formation, glycosylation, lipidation, acetylation, phosphorylation, or any other operations, such as conjugation with a labeling component.

In one embodiment of the present invention, the antibody or the fragment of the present invention is glycosylated with an engineered yeast N-linked glycan or CHO N-linked glycan.

In certain embodiments, the antibody disclosed herein can be further modified to comprise other non-protein portions known in the art and readily available. Suitable portions for antibody derivatization include, but are not limited to, water-soluble polymers. Non-limiting examples of water-soluble polymers include, but are not limited to, polyethylene glycol (PEG), ethylene glycol/propylene glycol copolymer, carboxymethyl cellulose, glucan, polyvinyl alcohol, polyvinylpyrrolidone, poly-1,3-dioxane, poly-1,3,6-trioxane, ethylene/maleic anhydride copolymer, polyamino acid (homopolymer or random copolymer), and glucan or poly(n-vinylpyrrolidone), polyethylene glycol, propylene glycol homopolymer, polypropylene oxide/ethylene oxide copolymer, polyoxyethylated polyol (such as glycerol), polyvinyl alcohol, and mixtures thereof.

In some embodiments, the modifications to the antibody or the fragment thereof described herein is PEGylation. An antibody can be PEGylated to, e.g. increase the biological (e.g., serum) half-life of the antibody. As used herein, the term “polyethylene glycol” is intended to encompass any form of PEG which has been used to derivatize other proteins, such as mono (C₁-C₁₀) alkoxy- or aryloxy polyethylene glycol or polyethylene glycol-maleimide. In certain embodiments, the antibody to be PEGylated is an aglycosylated antibody. Methods for PEGylation of proteins are known in the art and can be applied to the antibody of the present invention, for example, see EP 0154316 and EP 0401384.

In some embodiments, the anti-PD-L1 antibody is a monoclonal antibody.

In some embodiments, the anti-PD-L1 antibody is humanized. Different methods for humanizing antibodies are known to those skilled, as summarized by Almagro & Fransson, the content of which is incorporated in its entirety herein by reference (Almagro J. C. and Fransson J (2008) Frontiers in Bioscience 13:1619-1633).

In some embodiments, the anti-PD-L1 antibody is a human antibody. The human antibody can be prepared using a variety of techniques known in the art. The human antibody is generally described in van Dijk and van de Winkel, Curr. Opin. Pharmacol 5:368-74(2001) and Lonberg, Curr. Opin. Immunol 20:450-459(2008).

In some embodiments, the anti-PD-L1 antibody is a chimeric antibody.

In some embodiments, at least a portion of the framework sequence of the anti-PD-L1 antibody is a human consensus framework sequence. In one embodiment, the anti-PD-L1 antibody of the present invention also comprises an antibody fragment thereof, preferably an antibody fragment selected from: Fab, Fab′, Fab′-SH, Fv, a single-chain variable fragment (e.g., scFv), or (Fab′)₂; a single-domain antibody; a diabody (dAb), and linear antibody.

In certain embodiments, the anti-PD-L1 antibody molecule is in the form of a bispecific or multispecific antibody molecule. The multispecific antibody molecule, for example, may be a trispecific antibody molecule, and comprises a first binding specificity for PD-L1 and second and third binding specificities for one or more molecules.

### III. Immunoconjugates

In some embodiments, the present invention further provides an anti-PD-L1 antibody (“an immunoconjugate”) conjugated to other substances. In some embodiments, the other substance is, for example, a therapeutic agent or marker, such as a cytotoxic agent or an immunosuppressive or chemotherapeutic agent. The cytotoxic agent includes any agent that is harmful to cells.

Antibodies can also be attached to a solid phase support, which is particularly useful for immunoassays or purification of target antigens. Such solid phase supports include, but are not limited to, glass, cellulose, polyacrylamide, nylon, polystyrene, polyvinyl chloride, or polypropylene.

In some embodiments, the immunoconjugate is used to prevent or treat a tumor. In some embodiments, the tumor is cancer. In some embodiments, the immunoconjugate is used to prevent or treat an infection.

### IV. Nucleic Acid of the Present Invention and Host Cell Comprising Same

In one aspect, the present invention provides a nucleic acid encoding any of the above antibodies or the fragments thereof or any one of chains thereof. In one embodiment, provided is a vector comprising the nucleic acid. In one embodiment, the vector is an expression vector. In one embodiment, provided is a host cell comprising the nucleic acid or the vector. In one embodiment, the host cell is eukaryotic. In another embodiment, the host cell is selected from a yeast cell, a mammal cell (e.g., CHO cell or 293 cell), and other cells suitable for preparing an antibody or an antigen-binding fragment thereof. In another embodiment, the host cell is prokaryotic.

For example, the nucleic acid of the present invention comprises a nucleic acid encoding an amino acid sequence selected from any one of SEQ ID NOs: 14 to 18 and SEQ ID NOs: 20 to 24, or a nucleic acid encoding an amino acid sequence having at least 85%, 90%, 91%, 92%, 93%, 94%, 95%, 96%, 97%, 98%, or 99% identity to an amino acid sequence selected from any one of SEQ ID NOs: 14 to 18 and SEQ ID NOs: 20 to 24.

The present invention further provides a nucleic acid that is hybridized under a stringent condition with the following nucleic acids or a nucleic acid having one or more substitutions (e.g., conservative replacements), deletions or insertions as compared to the following nucleic acids: a nucleic acid comprising a nucleic acid sequence encoding an amino acid sequence selected from any one of SEQ ID NOs: 14 to 18 and SEQ ID NOs: 20 to 24; or a nucleic acid comprising a nucleic acid sequence encoding an amino acid sequence having at least 85%, 90%, 91%, 92%, 93%, 94%, 95%, 96%, 97%, 98%, or 99% identity to an amino acid sequence selected from any one of SEQ ID NOs: 14 to 18 and SEQ ID NOs: 20 to 24.

In one embodiment, provided are one or more vectors comprising the nucleic acid. In one embodiment, the vector is an expression vector, such as a eukaryotic expression vector. The vector includes, but is not limited to, a virus, a plasmid, a cosmid, a lambda phage, or a yeast artificial chromosome (YAC). In one embodiment, the vector is pTT5 vector.

Once the expression vector or DNA sequence has been prepared for expression, the expression vector can be transfected or introduced into suitable host cells. Various techniques can be used for this purpose, for example, protoplast fusion, calcium phosphate precipitation, electroporation, retroviral transduction, viral transfection, biolistics, lipid-based transfection, or other conventional techniques. In the case of protoplast fusion, cells are incubated in a culture medium and screened for appropriate activity. Methods and conditions for incubating the resulting transfected cell and for isolating the resulting antibody molecules are known to those skilled in the art and may be varied or optimized according to the particular expression vector and the particular mammalian host cell used based on the present description and methods known in the art.

Additionally, cells having stably incorporated DNA in chromosomes thereof can be selected by introducing one or more markers permitting the selection of transfected host cells. The markers may, for example, provide prototrophy, biocidal (e.g., antibiotics) resistance, or heavy metal (e.g., copper) resistance, etc., for an auxotrophic host. Selectable marker genes may be linked directly to a DNA sequence to be expressed or introduced through co-transformation into the same cell. Additional elements may also be required for optimal synthesis of mRNA. The elements may include splicing signals, transcriptional promoters, enhancers, and termination signals. In one embodiment, provided is a host cell comprising one or more polynucleotides of the present invention. In some embodiments, provided is a host cell comprising the expression vector of the present invention. In some embodiments, the host cell is selected from a yeast cell, a mammalian cell, or other cells suitable for preparing an antibody or an antigen-binding fragment thereof. Suitable host cells comprise prokaryotic microorganisms, such as E. coli. The host cells may also be eukaryotic microorganisms such as filamentous fungi or yeast, or various eukaryotic cells such as insect cells and the like. Vertebrate cells may also be used as hosts. For example, a mammalian cell line engineered to be suitable for suspension growth may be used. Examples of useful mammalian host cell lines include monkey kidney CV1 line (COS-7) transformed by SV40; human embryonic kidney line (HEK 293 or 293F cells), 293 cell, baby hamster kidney cell (BHK), monkey kidney cell (CV1), African green monkey kidney cell (VERO-76), human cervical cancer cell (HELA), canine kidney cell (MDCK), buffalo rat liver cell (BRL 3A), human lung cell (W138), human liver cell (Hep G2), Chinese hamster ovary cell (CHO cell), CHOS cell, NSO cell, myeloma cell line such as Y0, NS0, P3X63 and Sp2/0. For reviews of mammalian host cell lines suitable for protein production, see, for example, Yazaki and Wu, Methods in Molecular Biology, vol. 248 (edited by B. K. C. Lo, Humana Press, Totowa, N.J.), pp. 255-268 (2003). In one preferred embodiment, the host cell is a CHO cell or 293 cell, e.g., a HEK293 cell, e.g., HEK293-F.

### V. Production and Purification of the Antibody Molecule of the Present Invention

In one embodiment, the present invention provides a method for preparing an anti-PD-L1 antibody or a fragment thereof (preferably an antigen binding fragment), wherein the method comprises culturing host cells under conditions suitable for the expression of the nucleic acid encoding the antibody or the fragment thereof (preferably the antigen-binding fragment), and optionally isolating the antibody or the fragment thereof (preferably the antigen-binding fragment). In a certain embodiment, the method further comprises recovering the anti-PD-L1 antibody or fragment thereof (preferably antigen-binding fragment) from the host cells. In one embodiment, the present invention provides a method for preparing an anti-PD-L1 antibody, wherein the method comprises culturing host cells comprising a nucleic acid encoding the antibody (e.g., any one and/or more polypeptide chains) or an expression vector comprising the nucleic acid, as provided above, under conditions suitable for expressing antibodies, and optionally recovering the antibody from the host cells (or the host cell medium). For recombinant production of the anti-PD-L1 antibody, a nucleic acid encoding the antibody (e.g., the antibody described above, e.g., any one and/or more polypeptide chains) is isolated and inserted into one or more vectors for further cloning and/or expressing in the host cells. Such a nucleic acid can be easily isolated and sequenced by using conventional procedures (e.g., by using oligonucleotide probes that are capable of specifically binding to genes encoding heavy and light chains of antibodies).

The antibody molecule prepared as described herein can be purified by known prior art such as high performance liquid chromatography, ion exchange chromatography, gel electrophoresis, affinity chromatography, and size exclusion chromatography. The actual conditions used to purify a particular protein also depend on factors such as net charge, hydrophobicity, and hydrophilicity, and these will be apparent to those skilled in the art. The purity of the antibody molecule of the present invention can be determined by any one of a variety of well-known analytical methods including size exclusion chromatography, gel electrophoresis, high performance liquid chromatography, and the like.

### VI. Assays

The anti-PD-L1 antibody provided herein can be identified, screened, or characterized for its physical/chemical properties and/or biological activity through a variety of assays known in the art. In one aspect, the antigen-binding activity of the antibody disclosed herein is analyzed, for example, by known methods such as ELISA, and Western blotting. The binding to PD-L1 can be determined by methods known in the art, and exemplary methods are disclosed herein. In some embodiments, the binding of the antibody to PD-L1 is determined by SPR or bio-layer interferometry.

The present invention also provides an assay for identifying anti-PD-L1 antibodies having bioactivity. Biological activities may include, for example, binding to PD-L1 (e.g., binding to human PD-L1), binding to PD-L1 on the cell surface, and inhibition of binding to PD-1/PD-L1 or binding to PD-1/PD-L2. Further provided is an antibody having such biological activities in vivo and/or in vitro.

In certain embodiments, the antibody disclosed herein is characterized for such biological activities.

The present invention also provides methods for identifying properties of antibody PD-L1, such as druggability-related properties. Such druggability-related properties include, for example, thermal stability (e.g., long-term thermal stability) or solubility.

Cells for use in any of said in vitro assays include cell lines that naturally express PD-L1 or that are engineered to express PD-L1. Such cells also include cell lines that express PD-L1 and cell lines that do not normally express PD-L1 and have been transfected with DNA encoding PD-L1.

It will be appreciated that any of the above assays can be performed by using the immunoconjugate of the present invention in place of or in addition to the anti-PD-L1 antibody.

It will also be appreciated that any of the above assays can be performed on the anti-PD-L1 antibody and other active agents.

### VII. Pharmaceutical Compositions and Pharmaceutical Preparations

In some embodiments, the present invention provides a composition comprising any of the anti-PD-L1 antibodies or the fragments thereof (preferably the antigen-binding fragments), or the immunoconjugates thereof described herein, wherein, preferably, the composition is a pharmaceutical composition. In one embodiment, the composition further comprises pharmaceutical excipients. In one embodiment, the composition, e.g., the pharmaceutical composition, comprises the anti-PD-L1 antibody or fragment thereof of the present invention or the immunoconjugate thereof, and a combination of one or more other therapeutic agents (e.g., chemotherapeutic agents, cytotoxic agents, vaccines, other antibodies, active anti-infective agents, small molecule drugs, or immunomodulatory agents).

In some embodiments, the composition is used for preventing or treating a tumor. In some embodiments, the tumor is cancer. In some embodiments, the composition is used to prevent or treat an infection.

The present invention also includes a composition (including a pharmaceutical composition or a pharmaceutical preparation) comprising an anti-PD-L1 antibody or an immunoconjugate thereof, and/or a composition (including a pharmaceutical composition or a pharmaceutical preparation) comprising a polynucleotide encoding the anti-PD-L1 antibody. In certain embodiments, the compositions comprise one or more antibodies binding to PD-L1 or the fragments thereof, or one or more polynucleotides encoding the one or more antibodies that bind PD-L1 or the fragments thereof.

Such compositions can further comprise suitable pharmaceutical excipients, such as a pharmaceutical carrier known in the art, including buffers.

As used herein, the “pharmaceutical carrier” includes any and all solvents, dispersion media, isotonic agents and absorption delaying agents, and the like that are physiologically compatible. The pharmaceutical carrier suitable for use in the present invention can be sterile liquid, such as water and oil, including petroleum, or oil of an animal, vegetable, or an synthetic source, e.g., peanut oil, soybean oil, mineral oil, and sesame oil. Water is a preferred carrier when the pharmaceutical composition is administered intravenously. Saline solutions, aqueous dextrose and glycerol solutions can also be employed as liquid carriers, particularly for injectable solutions. Suitable excipients includes starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. For use and application of excipients, see Handbook of Pharmaceutical Excipients, 5^{th} ed, R. C. Rowe, P. J. Seskey and S. C. Owen, Pharmaceutical Press, London, Chicago. The composition may further comprise a small quantity of wetting agent, emulsifier, or pH buffer, if desired. The compositions may take the form of a solution, a suspension, an emulsion, a tablet, a pill, a capsule, a powder, a sustained release preparation, and the like. Oral preparations may comprise standard pharmaceutical carriers and/or excipients such as pharmaceutical-grade mannitol, lactose, starch, magnesium stearate and saccharin.

The pharmaceutical preparation, preferably in the form of a lyophilized preparation or an aqueous solution, comprising the anti-PD-L1 antibody described herein can be prepared by mixing the anti-PD-L1 antibody of desired purity of the present invention with one or more optional pharmaceutical excipients (Remington's Pharmaceutical Sciences, 16^{th} ed, Osol, A. (1980)).

An exemplary lyophilized antibody preparation is described in U.S. Pat. No. 6,267,958. The aqueous antibody preparation includes those described in U.S. Pat. No. 6,171,586 and WO2006/044908, and the latter preparation comprises a histidine-acetate buffer.

The pharmaceutical composition or preparation of the present invention may also comprise more than one active ingredient required by a treated particular indication, preferably active ingredients having complementarity activities without adversely affecting one another. For example, it may be also desirable to provide other anti-cancer or anti-infective active ingredients, such as chemotherapeutic agents, cytotoxic agents, vaccines, other antibodies, anti-infective active agents, small molecule drugs, or immunomodulatory agents.

A sustained release preparation can be formulated. Suitable examples of the sustained release preparation include a semipermeable matrix of a solid hydrophobic polymer containing an antibody. The matrix is in the form of a shaped article, such as a film or a microcapsule.

### VIII. Combination Product or Kit

In some embodiments, the present invention also provides a combination product comprising the antibody or the antigen-binding fragment thereof disclosed herein, or the immunoconjugate thereof, and one or more additional therapeutic agents (e.g., chemotherapeutic agents, other antibodies, cytotoxic agents, vaccines, anti-infective active agents, small molecule drugs or immunomodulatory agents).

In some embodiments, the combination product is used for preventing or treating a tumor. In some embodiments, the tumor is cancer. In some embodiments, the composition product is used to prevent or treat an infection.

In some embodiments, two or more of the ingredients in the combination product may be administered to a subject in combination, sequentially, separately or simultaneously.

In some embodiments, the present invention also provides a kit comprising the antibody, the pharmaceutical composition, the immunoconjugate or the combination product disclosed herein, and optionally a package insert directing administration.

In some embodiments, the present invention also provides a pharmaceutical product comprising the antibody, the pharmaceutical composition, the immunoconjugate, the combination product disclosed herein, optionally further comprising a package insert directing administration.

### IX. Uses of the Antibody Molecules of the Present Invention

In one aspect, the present invention relates to a method for modulating an immune response in an individual. The method comprises administering to a subject an effective amount of the antibody molecule (e.g., the anti-PD-L1 antibody) or the pharmaceutical composition or the immunoconjugate or the combination product or the kit disclosed herein, thereby modulating the immune response in the subject. In one embodiment, the antibody molecule (e.g., a therapeutically effective amount of the anti-PD-L1 antibody molecule) or the pharmaceutical composition or the immunoconjugate or the combination product or the kit disclosed herein restores, enhances, stimulates or increases the immune response in the subject.

In some embodiments, the present invention relates to a method for inhibiting the activity of PD-L1, blocking the binding of PD-1 to PD-L1, or blocking the binding of PD-1 to PD-L2 in an individual, comprising administering to a subject an effective amount of the antibody molecule (e.g., the anti-PD-L1 antibody) or the pharmaceutical composition or the immunoconjugate or the combination product or the kit disclosed herein.

In another aspect, the present invention relates to a method for preventing or treating a tumor (e.g., cancer) in a subject, wherein the method comprises administering to the subject an effective amount of the antibody molecule (e.g., the anti-PD-L1 antibody) or the pharmaceutical composition or the immunoconjugate or the combination product or the kit disclosed herein. In some embodiments, the tumor is cancer.

In another aspect, the present invention relates to a method for preventing or treating an infectious disease in a subject, wherein the method comprises administering to the subject an effective amount of the antibody molecule (e.g., the anti-PD-L1 antibody) or the pharmaceutical composition or the immunoconjugate or the combination product or the kit disclosed herein.

In another aspect, the present invention relates to a method for inducing antibody-dependent cell-mediated cytotoxicity in the subject, and the method comprises administering to the subject an effective amount of the antibody molecule (e.g., the anti-PD-L1 antibody) or the pharmaceutical composition or the immunoconjugate or the combination product or the kit disclosed herein.

The subject can be a mammal, e.g., a primate, preferably a higher primate, e.g., a human (e.g., a patient having or at risk of having the disease described herein). In one embodiment, the subject has or is at risk of having the disease described herein (e.g., the tumor or infectious disease as described herein). In certain embodiments, the subject is receiving or has received other therapies, e.g., chemotherapy and/or radiation therapy. Alternatively or in combination, the subject is immunocompromised due to infection or is at risk of being immunocompromised due to infection.

In some embodiments, the tumor described herein is, e.g., cancer.

In one embodiment, the disease, e.g., tumor, is a disease, e.g., a tumor such as cancer, with elevated (nucleic acid or protein) levels of PD-L1, PD-L2, or PD-1. In some embodiments, the tumor is a tumor that is capable of being inhibited by inhibiting the binding of PD-1 to PD-L1 or PD-L2, e.g., cancer. In some embodiments, the tumor or infection is a disease that would benefit from inhibition of nucleic acid or protein levels of PD-L1 or PD-1 or PD-L2. In some embodiments, the tumor or infection benefits from blocking the binding of PD-1 to PD-L1, or the binding of PD-1 to PD-L2.

In other aspects, the present invention provides uses of the anti-PD-L1 antibody or the fragment thereof or the immunoconjugate thereof or the combination product or the kit in the manufacture or preparation of a drug for prevention or treatment of the related diseases or conditions mentioned herein.

In some embodiments, the antibody or the fragment thereof, the immunoconjugate, the composition, the combination product, or the kit disclosed herein delays the onset of the conditions and/or symptoms associated with the conditions.

In some embodiments, the prevention or treatment method described herein further comprises administering to a subject or an individual the antibody molecule (e.g., the anti-PD-L1 antibody or the fragment thereof) or the pharmaceutical composition or the immunoconjugate or the combination product or the kit disclosed herein in combination with one or more other therapies, e.g., therapeutic modalities and/or other therapeutic agents.

In some embodiments, the therapeutic modality includes a surgical treatment or a radiation therapy. In some embodiments, the therapeutic agents are selected from chemotherapeutic agents, cytotoxic agents, vaccines, other antibodies, anti-infective active agents, small molecule drugs, or immunomodulatory agents.

Combination therapy encompasses combined administration (where two or more therapeutic agents are contained in the same kit or formulation, or separate kits or formulations), and separate administration, in which administration of the antibody or the immunoconjugate of the present invention can occur prior to, concurrently with, and/or after administration of the other therapies, e.g., therapeutic modalities and/or therapeutic agents. The antibody molecule and/or other therapies, e.g., therapeutic agents or therapeutic modalities, can be administered during active diseases or in the period of remission or less active diseases. The antibody molecule may be administered prior to other therapies, concurrently with other therapies, after other therapies, or during remission of diseases.

In some embodiments, the antibody combinations described herein can be administered separately (e.g., as separate antibodies) or in linkage (e.g., as a bispecific or trispecific antibody molecule). The antibody of the present invention (the pharmaceutical composition or the immunoconjugate comprising the same, and any other therapeutic agents) can be administered by any suitable method, including parenteral administration, intrapulmonary administration, intranasal administration, and intralesional administration if required by locoregional treatment. Parenteral infusion includes intramuscular, intravenous, intra-arterial, intraperitoneal, or subcutaneous administration. The medicaments may be administered by any suitable means, such as injection, e.g., intravenous or subcutaneous injection, to some extent depending on short-term or long-term treatment. Various administration schedules are encompassed herein, including, but not limited to, single administration or multiple administrations at multiple time points, bolus injection, and pulse infusion.

In order to prevent or treat diseases, the appropriate dosage of the antibody of the present invention (when used alone or in combination with one or more other additional therapeutic agents) will depend on types of diseases to be treated, types of antibodies, severity and progression of the disease, purpose of administration (prophylactic or therapeutic), previous treatments, clinical histories of patients, responses to the antibody, and the discretion of an attending physician. The antibody is suitably administered to a patient through a single treatment or through a series of treatments.

The dosage and regimen of the anti-PD-L1 antibody molecule can be determined by those skilled. It will be appreciated that any treatment can be performed using the immunoconjugate or the composition or the combination product or the kit disclosed herein instead of or in addition to the anti-PD-L1 antibody.

### X. Methods and Compositions for Diagnosis and Detection

In certain embodiments, any of the anti-PD-L1 antibodies or the antigen-binding fragments thereof provided herein can be used for detection of the presence of PD-L1 in a biological sample. The term “detection” used herein includes quantitative and qualitative detections, and exemplary detections may involve immunohistochemistry, immunocytochemistry, flow cytometry (e.g., FACS), magnetic beads complexed with antibody molecules, ELISA, and PCR (e.g., RT-PCR). In certain embodiments, the biological sample is blood, serum, or other liquid samples of biological origin. In certain embodiments, the biological sample includes cells or tissues. In some embodiments, the biological sample is derived from a proliferative or cancerous lesion. In one embodiment, provided is an anti-PD-L1 antibody for use in a diagnostic or detection method. In another aspect, provided is a method for detecting the presence of PD-L1 in a biological sample. In certain embodiments, the method comprises detecting the presence of PD-L1 proteins in a biological sample. In certain embodiments, the method comprises detecting the presence of a nucleic acid associated with the PD-L1 gene in a biological sample. In certain embodiments, the PD-L1 is human PD-L1. In certain embodiments, the method comprises contacting the biological sample with the anti-PD-L1 antibody as described herein in a condition that allows the anti-PD-L1 antibody to bind to PD-L1, and detecting whether a complex is formed by the anti-PD-L1 antibody and PD-L1. The formation of the complex indicates the presence of PD-L1. The method may be an in vitro or in vivo method. In one embodiment, the anti-PD-L1 antibody is used to select a subject suitable for treatment with the anti-PD-L1 antibody, e.g., wherein PD-L1 is a biomarker for selecting the subject.

In one embodiment, the antibody of the present invention can be used to diagnose cancers or tumors, e.g., to assess (e.g., monitor) the treatment or progression, diagnosis and/or staging of a disease (e.g., the hyperproliferative or cancerous disease) described herein in a subject. In certain embodiments, provided is a labeled anti-PD-L1 antibody.

In some embodiments of the invention provided herein, the sample is obtained prior to treatment with the anti-PD-L1 antibody. In some embodiments, the sample is obtained prior to treatment with an anti-cancer drug. In some embodiments, the sample is obtained after the cancer has metastasized. In some embodiments, the sample is a formalin-fixed, paraffin-embedded (FFPE) sample. In some embodiments, the sample is a biopsy (e.g., a core biopsy) specimen, a surgical specimen (e.g., a specimen from a surgical resection), or a fine-needle aspirate.

In some embodiments, PD-L1 is detected prior to treatment, e.g., prior to initial treatment or prior to a treatment after an interval from a certain treatment.

In some embodiments, provided is a method for treating a tumor or infection, which comprises: detecting the presence of PD-L1 in a subject (for example, using a sample, e.g., a sample containing cancer cells of the subject), thereby determining a PD-L1 value; comparing the PD-L1 value to a control value (e.g., the PD-L1 value of the sample of a healthy individual); and if the PD-L1 value is greater than the control value, administering a therapeutically effective amount of an anti-PD-L1 antibody (e.g., the anti-PD-L1 antibody described herein), optionally in combination with one or more of other therapies, to the subject, thereby treating the tumor or infection.

It can be understood that the various embodiments described in various parts of the present invention, e.g., diseases, therapeutic agents, therapeutic modalities, administration and the like, are equally applicable to, or may be combined with, embodiments of other parts of the present invention. The description in the various parts of the present invention applies to the properties, the uses, the method of the antibody molecule, and equally to the compositions, conjugates, combination products and kits comprising the antibody.

### EXAMPLES

### Example 1: Design and Construction of a Single-Domain Antibody Variable Region Mutant Library

Design of a Single-Domain Antibody Variable Region Mutant Library

The amino acid sequences of the CDR1, CDR2 and CDR3 of the existing single-domain antibody HzNB1613 (SEQ ID NO: 14 in CN 107686520 A, hereinafter AmNB1613.0, numbered according to Chothia, the CDR regions being defined by the ABM rules) were subjected to the mutations shown in following Tables to construct a mutant library.

TABLE 1
Design of CDR1 mutant library IBYDL019

| | Amino acid | Codons for mutant | |
|---|---|---|---|
| Sites | residues | amino acids | Diversity |
| 26 | Ala(A) | NNK | 20 |
| 27 | Tyr(Y) | NNK | 20 |
| 28 | Thr(T) | NNK | 20 |
| 29 | Ile(I) | NNK | 20 |
| 30 | Ser(S) | NNK | 20 |
| 31 | Arg(R) | NNK | 20 |
| 32 | Asn(N) | NNK | 20 |
| 33 | Ser(S) | NNK | 20 |
| 34 | Met(M) | NNK | 20 |
| 35 | Gly(G) | G (mutant amino acid) | 1 |

Based on the amino acids in the CDR1 shown in Table 1, the bases encoding the original amino acids at each site were designed as follows. For example, the codon for Ala at site 26 is GCC, in the mutation of the first position, G accounts for 80%, and the remaining 20% is equally divided by A/C/T; in the mutation of the second position, C accounts for 80%, the remaining 20% is equally divided by A/G/T; and in the mutation of the third position K, G/T accounts for 50% (N: A/C/G/T, K: G/T). Therefore, the theoretical diversity of the CDR1 mutant library IBYDL019 is 20⁹≈5.1×10¹¹.

TABLE 2
CDR2 mutant library IBYDL020

| | Amino acid | Mutant | |
|---|---|---|---|
| Sites | residues | amino acids | Diversity |
| 50 | Ala(A) | A | 1 |
| 51 | Ile(I) | NNK | 20 |
| 52 | Glu(E) | NNK | 20 |
| 53 | Ser(S) | NNK | 20 |
| 54 | Asp(D) | NNK | 20 |
| 55 | Gly(G) | NNK | 20 |
| 56 | Ser(S) | NNK | 20 |
| 57 | Thr(T) | NNK | 20 |
| 58 | Ser(S) | S | 1 |

Based on the amino acids in the CDR2 shown in Table 2, the ratio of the bases encoding the original amino acid at each site is the same as that of the CDR1 mutant library, and the theoretical diversity of the CDR2 mutant library IBYDL020 is 20⁷≈1.3×10⁹.

TABLE 3
CDR3 mutant library IBYDL021-023

| | Amino acid | | |
|---|---|---|---|
| Sites | residues | Mutant amino acids | Diversity |
| 95 | Pro(P) | NNK | 20 |
| 96 | Lys(K) | NNK | 20 |
| 97 | Val(V) | NNK | 20 |
| 98 | Gly(G) | NNK | 20 |
| 99 | Leu(L) | NNK | 20 |
| 100 | Gly(G) | NNK | 20 |
| 100A | Pro(P) | NNK | 20 |
| 100B | Arg(R) | NNK | 20 |
| 100C | Thr(T) | NNK | 20 |
| 100D | Ala(A) | NNK | 20 |
| 100E | Leu(L) | NNK | 20 |
| 100F | Gly(G) | NNK | 20 |
| 100G | His(H) | NNK | 20 |
| 100H | Leu(L) | NNK | 20 |
| 100I | Ala(A) | NNK | 20 |
| 100J | Phe(F) | NNK | 20 |
| 100K | Met(M) | NNK | 20 |
| 100L | Thr(T) | NNK | 20 |
| 100M | Leu(L) | NNK | 20 |
| 100N | Pro(P) | NNK | 20 |
| 100O | Ala(A) | NNK | 20 |
| 100P | Leu(L) | NNK | 20 |
| 101 | Asn(N) | NNK | 20 |
| 102 | Tyr(Y) | NNK | 20 |

Based on the amino acids in the CDR3 shown in Table 3, the CDR3 mutant library is split into 3 for construction due to too long amino acids in the CDR3. The theoretical diversity of the amino acid library IBYDL021 between the sites 95 and 100B is 20⁸≈1.3×10¹⁰, the theoretical diversity of the amino acid library IBYDL022 between the sites 100C and 100J 20⁸≈1.3×10¹⁰, and the theoretical diversity of the amino acid library IBYDL023 between the sites 100K and 102 is 20⁸≈1.3×10¹⁰.

Construction of a Single-Domain Antibody Variable Region Mutant Library

The gene sequence of the single-domain antibody HzNB1613 was placed between two BamHI enzyme cutting sites in the yeast surface display plasmid pYDC011 (SEQ ID NO: 26) and displayed on the yeast surface as a parent control before affinity maturation.

The method comprises the following specific steps: 1. the synthetic genes of HzNB1613 (SEQ ID NO: 14 in CN 107686520 A, Suzhou Hongxun Biotechnology Co., Ltd.) were used as templates for amplification with primers AMP0083 and AMP0084; 2. plasmid pYDC011 was digested with BamHI (New England Biolab, Catalog No. R3136V), followed by gel extraction (QIAGEN Gel Extraction Kit, Catalog No. 28704); 3. the amplified product and digested product were extracted from 1% agarose gel; 4. after extraction, the products were homologously recombined in vitro using One Step Cloning Kit (Vazyme Catalog No. C113-02) according to the product manual; 5. the recombined product was transferred into E. coli Top10 competent cells (Tiangen Biotech (Beijing) Co., Ltd., Catalog No. CB104-02), and the cells were coated on an ampicillin-resistant LB plate and cultured at 37° C. overnight; 6. after the growing monoclonal colonies were verified by sequencing, the correct plasmid was named as pYDC012.

The required primers were designed according to the library construction schemes of Tables 1, 2 and 3 and synthesized by Genewiz Biological Technology Co., Ltd. The sequences are shown in the sequence listing.

IBYDL019 library DNA amplification: 1. fragment 019-F was amplified with primers AMP0090 and AMP0082 using pYDC012 as a template; 2. fragment 019-R was amplified with primers AMP0085 and AMP0044 using pYDC012 as a template; 3. fragments 019-F and 019-R were extracted from gel and used as a PCR amplification template to amplify the full-length fragment 019 with primers AMP0082 and AMP0044.

IBYDL020 library DNA amplification: 1. fragment 020-F was amplified with primers AMP0091 and AMP0082 using pYDC012 as a template; 2. fragment 020-R was amplified with primers AMP0086 and AMP0044 using pYDC012 as a template; 3. fragments 020-F and 020-R were extracted from gel and used as a PCR amplification template to amplify the full-length fragment 020 with primers AMP0082 and AMP0044.

IBYDL021 library DNA amplification: 1. fragment 021-F was amplified with primers AMP0092 and AMP0082 using pYDC012 as a template; 2. fragment 021-R was amplified with primers AMP0087 and AMP0044 using pYDC012 as a template; 3. fragments 021-F and 021-R were extracted from gel and used as a PCR amplification template to amplify the full-length fragment 021 with primers AMP0082 and AMP0044.

IBYDL022 library DNA amplification: 1. fragment 022-F was amplified with primers AMP0093 and AMP0082 using pYDC012 as a template; 2. fragment 022-R was amplified with primers AMP0088 and AMP0044 using pYDC012 as a template; 3. fragments 022-F and 022-R were extracted from gel and used as a PCR amplification template to amplify the full-length fragment 022 with primers AMP0082 and AMP0044.

IBYDL023 library DNA amplification: 1. fragment 023-F was amplified with primers AMP0094 and AMP0082 using pYDC012 as a template; 2. fragment 023-R was amplified with primers AMP0089 and AMP0044 using pYDC012 as a template; 3. fragments 023-F and 023-R were extracted from gel and used as a PCR amplification template to amplify the full-length fragment 023 with primers AMP0082 and AMP0044.

100 μg of plasmid pYDC011 was digested with BamHI, and extracted using a PCR product recovery kit (QIAGEN PCR Purification Kit, Catalog No. 28104) to give a sufficient number of linear plasmids. The linear plasmids and library DNAs as obtained above were mixed at a ratio of 4 μg:12 μg. The mixture of each library and linear plasmid was electrotransfected into EBY100 yeast strain purchased from ATCC Number: MYA-4941™ according to the existing method (Lorenzo Benatuil et al., An improved yeast transformation method for the generation of very large human antibody libraries. Protein Engineering, Design & Selection vol. 23, no. 4, pp. 155-159, 2010). After electrotransfection, the libraries were gradiently diluted and coated on SD-Trp (TAKARA, Catalog No. 630309) plates. The number of growing colonies was counted. The actual diversity of the library IBYDL019 obtained was: 4.0×10⁸, IBYDL020: 3.0×10⁸, IBYDL021: 2.8×10⁸, IBYDL022: 3.9×10⁸ and IBYDL023: 3.7×10⁸.

### Example 2: Screening of a Single-Domain Antibody Mutant Library and Differential Staining of Mutants

Screening of a Single-Domain Antibody Mutant Library

2.0×10⁹ yeast cells were taken for culturing and inducing based on yeast display-based HzNB1613 affinity-matured mutant (referred to as AmNB1613^{Mutant}) libraries IBYDL019, IBYDL020, IBYDL021, IBYDL022, and IBYDL023.

Magnetic-activated cell sorting was performed using the MACS system from Miltenyi in a first round of screening, 2×10⁹ yeast cells were incubated in FACS buffer (1×PBS, containing 1% bovine serum albumin) supplemented with 10 nM PD-L1 Biotin (Acro Biosystems, PD1-H82E5) for 30 min at room temperature. The cells were washed once with 50 mL of pre-cooled FACS buffer (lx PBS, containing 1% bovine serum albumin) and resuspended with 10 mL of the same buffer, followed by addition of 40 μL of streptavidin microbeads (Miltenyi LS) and incubation at 4° C. for 15 min. The mixture was centrifuged at 3000 rpm for 3 min in a centrifuge. After discarding the supernatant, the cells were resuspended in 10 mL of FACS washing buffer. The resulting cell suspension was loaded on a Miltenyi LS column. After loading, the column was washed three times, with 3 mL of FACS buffer each time. The Miltenyi LS column was removed from the magnetic field and eluted with 5 mL of growth medium, and the eluted yeast cells were collected and placed in a culture flask, incubated overnight at 30° C. and induced by shaking at 20° C. for 24 h using growth medium. 1.5×10⁹ yeast cells were taken from each library for a second round of magnetic bead enrichment, and the cells were incubated in FACS buffer containing 1 nM PD-L1 Biotin at room temperature for 30 min.

The second round of magnetic bead enrichment was performed in the same way as the first round; specifically, the eluted yeast cells were collected and placed in a culture flask, incubated overnight at 30° C. and induced by shaking at 20° C. for 24 h using growth medium.

A third round of sorting on each library cell after two rounds of magnetic bead enrichment using a flow cytometer, comprises the following specific steps:

7.5×10⁷ yeast cells taken from each library were washed three times with FACS buffer (lx PBS, containing 1% bovine serum albumin), and then FACS buffer containing PD-L1 Biotin (1 nM) and Anti Flag (Sigma, Catalog No. F1804) antibody (diluted at a ratio of 1:1000, the same below) was added and the cell were incubated at room temperature for 30 min. After the cells were washed twice with FACS buffer, the cells were mixed with FACS buffer containing streptavidin (SA-PE, eBioscience, Catalog No. 12-4317-87, diluted at a ratio of 1:200, the same below), goat anti-mouse conjugated with Alex flow-647 (Thermo Fisher, Catalog No. A21235, diluted at a ratio of 1:200, the same below), and incubated in the absence of light at 4° C. for 15 min. The cells were washed twice with pre-cooled FACS washing buffer, resuspended in 2 mL of buffer, and transferred into a separator tube with a filter. Cells were sorted using the MoFlo_XDP ultra-rapid flow cytometric sorting system, and the sorted yeast cells were incubated overnight at 30° C.

A fourth round of the sorting was identical to the third round, the concentration of PD-L1 Biotin in the FACS buffer was reduced to 0.5 nM, and after four rounds of sorting, the monoclones were picked and sent for sequencing.

Several clones containing single mutant sequence were obtained by four rounds of sorting using PD-L1 Biotin. The antibodies contained in these clones were sequenced.

Differential Staining of AmNB1613^{mutant}

According to the sequencing result, the clone containing the sequence of the single cysteine and the N-linked glycosylation site was removed, then the remaining monoclonal yeast cells were shaken and induced at 20° C. for 24 h to display the AmNB1613^{Mutant}, and the AmNB1613^{Mutant} was stained with PD-L1 Biotin, and the specific steps were as follows:
1. 1×10⁶ cells were taken from each clone, washed once with FACS buffer, and added to 96-well U-bottom plates (Costar, Catalog No. CLS3799-50EA) at 1×10⁵ cells per well.
2. 100 μL of PD-L1 Biotin and Anti Flag antibody was added, the PD-L1 Biotin was 3-fold diluted in gradient from a maximum concentration of 100 nM for a total of 7 dilution gradients, negative control (0 nM) was added, and the cells were incubated at room temperature for 30 min;
3. the mixture was centrifuged at 3000 rpm at 4° C. for 3 min, and washed with pre-cooled FACS buffer twice;
4. 100 μL of FACS buffer containing SA-PE and goat anti-mouse conjugated with Alex Flour-647 was added, and the mixture was incubated in the absence of light on ice for 20 min;
5. after being washed twice with pre-cooled FACS buffer, the cells were resuspended in 100 μL of buffer, and assayed by a flow cytometer (BD, ACCURI C6);
6. the EC₅₀ values of antigens and single-domain antibodies were obtained using the antigen concentration as the abscissa and SA-PE median fluorescence value as the ordinate.

Based on the EC₅₀ values of the clones and their homology to the parental sequences, 2 clones (AmNB1613.1 and AmNB1613.12) were selected from IBYDL019 and 3 clones (AmNB1613.25, AmNB1613.28, and AmNB1613.36) were selected from IBYDL023, the sequences of which were shown in the sequence listing for further study. The cloned antibody genes were loaded onto expression vectors to express protein for subsequent identifications.

### Example 3: Fusion Expression of AmNB1613^{mutant}-FC

Expression and Purification of AmNB1613^{Mutant}-FC Protein

In order to prolong the half-life of the single-domain antibody and enhance the binding force of the single-domain antibody to cell surface antigens, the anti-PD-L1 single-domain antibody was fused and expressed with human IgG1 LALA Fc (SEQ ID NO: 25) using a molecular cloning method. Each nucleic acid encoding the gene sequence of AmNB1613^{Mutant}-FC was constructed into a vector of pTT5 to obtain an expression plasmid, and the vector containing the gene encoding the fusion protein was transferred into HEK293-F (Invitrogen, Catalog No. R79007) cells using a chemical transfection method, and 293F cells (Invitrogen) were passaged according to a desired transfection volume. The specific steps are as follows:

a. the cell culture was centrifuged the day before transfection to obtain a cell precipitate, the cells were suspended in a fresh Expi293 cell culture medium and the cell density was adjusted to 1.5×10⁶ cells/mL. HEK293 cells were further cultivated such that the cell density on the day of transfection was about 3×10⁶ cells/mL. Opti-MEM medium (Gibco, Catalog No. 31985-070) that was 1/10 (v/v) of the final volume of HEK293 cell suspension was used as a transfection buffer, 1 μg of the above prepared expression plasmid was added into the transfection buffer, mixed well, and filtered with a 0.22 μm filter for later use. An appropriate amount of polyethylenimine (PEI) (Polysciences, 23966) was added to the plasmids from the previous step (the mass ratio of plasmids to PEI was 1:3 in 293F cells), mixed well and incubated at room temperature for 10 min to give a DNA/PEI mixture. The DNA/PEI mixture was gently poured into HEK293 cell suspension, mixed well, and cultured at 37° C. and 8% CO₂ for 24 h, followed by the addition of VPA (Sigma, Catalog No. P4543-100G) to reach a final concentration of 2 mM. Then 2% (v/v) Feed (1 g/L Phytone Peptone+1 g/L Difco Select Phytone) was added and the resulting mixture was cultivated for 3 days, the culture was centrifuged at 13000 rpm for 20 min, and the supernatant was collected for subsequent assay of the expression yield and affinity of the supernatant sample in Example 4.

Purification of AmNB1613^{Mutant}-FC Protein

After cells were transfected as above, the resulting mixture was cultivated for 6 days after the addition of VPA and Feed, the cell culture medium was centrifuged at 13000 rpm for 20 min, and the supernatant was collected and purified by a pre-packed column Hitrap Mabselect Sure (GE, 11-0034-95). The procedures were as follows: the packing column was equilibrated with 5-fold column volume of equilibration buffer (0.2 M Tris, 1.5 M NaCl, pH 7.2) before purification; the collected supernatant was passed through the column, and then the column was washed with 10-fold column volume of equilibration buffer to remove non-specific binding proteins; the packing was washed with 5-fold column volume of elution buffer (1 M sodium citrate, pH 3.5), and the eluent was collected. 80 μL of Tris (2 M Tris) was added per 1 mL of eluent, and the mixture was exchanged into PBS buffer (Gibco, 70011-044) using an ultrafiltration concentration tube (Shanghai Tuokai Biotechnology Co., Ltd., MCPM02C67), and then the concentration was determined. 100 μg of purified protein was taken with its concentration adjusted to 1 mg/mL. The protein purity was determined by gel filtration column (TOSOH, Catalog No. 18675).

### Example 4: Assay for the Expression Yield and Affinity of AmNB1613^{mutant}-FC

Assay for Expression Yield and Affinity of AmNB1613^{Mutant}-FC Supernatant Sample

After the cells were transfected and then cultured for 3 days as shown in Example 3, the supernatant was collected and assayed for the yield of fusion expression of 5 affinity-matured mutants of the present invention, i.e., AmNB1613^{mutant}-FC and equilibrium dissociation constant (K_{D}) of the mutants binding to the antigen human PD-L1 (Acro Biosystems, Catalog No. PD1-H5229) using bio-layer interferometry (BLI).

A BLI affinity assay was conducted according to the existing method (Estep, P et al., High throughput solution Based measurement of antibody-antigen affinity and epitope binding. MAbs, 2013.5(2): p. 270-8) known in the art. Briefly, an AHQ sensor (ForteBio, 18-5060) was immersed in an assay buffer (PBS 1×, BSA 0.1%, Tween 20 0.05%) for 20 min before the assay; according to the method established by Estep, P et al., the affinity of the candidate AmNB1613^{mutant}-FC for PD-L1 was assayed with Octet Red96 (ForteBio): first, the baseline was established by detecting online for 120 s; then the supernatant of AmNB1613^{mutant}-FC and HzNB1613 was immobilized to the AHQ sensor (ForteBio, 18-5060); the immobilized sensor was exposed to a solution containing 100 nM human PD-L1 (Acro Biosystems, Catalog No. PD1-H5229) until to a plateau (100 s), and then transferred to an assay buffer for dissociation for at least 2 min. The background-corrected association and dissociation curves were fitted by the Octet analysis software (ForteBio) to generate the binding rate constant (kon) and dissociation rate constant (kdis), which were then used to calculate the equilibrium dissociation constant (K_{D}). Kinetic analysis was performed on the assay results using a 1:1 binding model.

In the assay described above, the expression yield of the selected 5 AmNB1613^{mutant}-FC supernatant samples expressed by HEK293-F and the affinities (K_{D} values) of the AmNB1613^{mutant}-FC for the antigens thereof are shown in Table 4.

TABLE 4
Expression yield and affinity of AmNB1613^{Mutant}-FC
supernatant samples

| | Concentration | K_{D} |
|---|---|---|
| Mutant ID | (μg/mL) | (Affinity M) |
| HzNB1613 | 21.7 | 1.64E−08 |
| AmNB1613.1 | 473.6 | 2.72E−09 |
| AmNB1613.12 | 297.4 | 2.26E−09 |
| AmNB1613.25 | 18.5 | 2.15E−09 |
| AmNB1613.28 | 23.7 | 2.67E−09 |
| AmNB1613.36 | 26.9 | 1.76E−09 |

Assay for Expression Yield and Affinity of AmNB1613^{Mutant}-FC Purified Samples

These 4 clones were further subjected to expression and purification as shown in Example 3, the antibody expression yield and affinity were assayed using the obtained antibody solution (as described above), and the results are shown in Table 5. As shown in Table 5, the antibody of the present invention has higher expression yield than the parent antibody, and the affinity thereof is significantly improved compared to the parent antibody, which is worthy of further development.

TABLE 5
Expression yield and affinity of AmNB1613^{mutant}-FC
purified samples

| | | K_{D} | Binding | |
|---|---|---|---|---|
| | Conc. | (Affinity | constant | Dissociation |
| Mutant ID | (ug/mL) | M) | (1/Ms) | constant (1/s) |
| HzNB1613 | 49 | 4.30E−08 | 8.05E+04 | 3.46E−03 |
| AmNB1613.1 | 189.8 | 8.14E−09 | 1.28E+05 | 1.04E−03 |
| AmNB1613.12 | 175.3 | 6.72E−09 | 1.39E+05 | 9.32E−04 |
| AmNB1613.25 | 194.4 | 6.87E−09 | 1.29E+05 | 8.87E−04 |
| AmNB1613.28 | 194.3 | 6.87E−09 | 1.13E+05 | 7.73E−04 |
| AmNB1613.36 | 200.1 | 4.21E−09 | 1.67E+05 | 7.02E−04 |

### Example 5: Detection of the Binding Level of AmNB1613^{mutant}-FC to Cell Surface Antigen

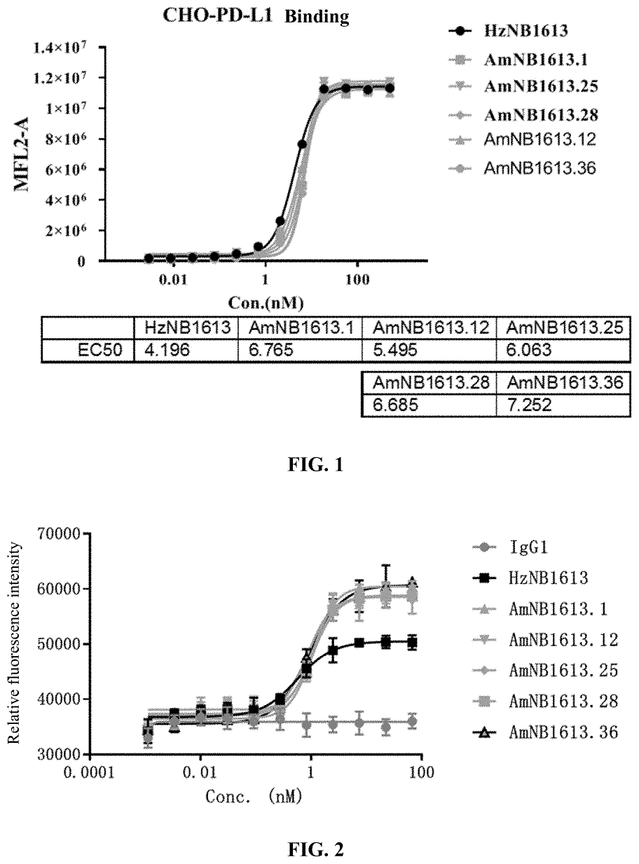

Over-expressing human PD-L1 antigen on the CHO cell surface and detecting the binding level of AmNB1613^{mutant}-FC to PD-L1, comprise the following steps:
1. Cell preparation: by using the ExpiCHO™ Expression System Kit (Invitrogen, Catalog No. A29133) according to the product manual of the manufacturer, the pCHO1.0 vector (Invitrogen) carrying human PD-L1 cDNA (Sino Biological Inc.) cloned to multiple cloning sites (MCSs) was transfected into Chinese hamster ovary cancer cells (CHO) (Invitrogen) to give CHO cells overexpressing human PD-L1 cells (CHO-PD-L1 cells); the CHO-PD-L1 cells were counted, diluted to 2×10⁶ cells/mL with a cell culture medium, and added to a U-bottom 96-well plate at 100 μL/well.
2. Cell staining: the cell suspension was centrifuged at 400 g on a centrifuge for 5 min to remove the cell medium, 100 μL of gradiently diluted 5 AmNB1613^{mutant}-FCs prepared and purified as described above, and HzNB1613 as a control, were added per well of a U-bottom 96-well plate, with a total of 12 gradients of 3-fold gradient dilution from a maximum concentration of 500 nM, and incubated for 30 min at room temperature; the plate was put onto the ice to stand for 30 min; the cell suspension was centrifuged at 400 g for 5 min, and then the supernatant was removed, and the cells were washed once with PBS to remove the unbound antibodies; 100 μL of PBS containing PE conjugated anti-human Fc antibody (Anti human Fc-PE antibody, Jackson Immuno Research, Catalog No. 2040-09) diluted at a ratio of 1:200 was added per well; the cells were incubated in the absence of light on the ice for 30 min, and the cell suspension was centrifuged at 400 g for 5 min to remove supernatant; the cells were washed with PBS to remove the unbound PE-conjugated anti-human Fc antibody; then the cells were resuspended with 100 μL of PBS, and the binding of the antibodies to cells was assayed by a flow cytometer (BD, ACCURI C6).
3. The binding curve of EC₅₀ values between antigen and single-domain antibody was plotted using the antigen concentration as the abscissa and SA-PE median fluorescence value as the ordinate, and the results are shown in FIG. 1.

It can be seen from FIG. 1, each AmNB1613^{mutant}-FCs (AmNB1613.1, AmNB1613.12, AmNB1613.25, AmNB1613.28, and AmNB1613.36) binds to PD-L1 antigen on the CHO cell surface at a level nearly identical to that of the parent antibody (HZnb1613), indicating that the affinity-matured mutants improve the overall K_{D} by a lower dissociation constant.

### Example 6: Detection of Inhibitory Effect of AmNB1613^{mutant}-FC on the Binding of PD-1/PD-L1 by MOA Assay

Anti-PD-1/PD-L1 antibodies can relieve the inhibitory effect on a downstream NFAT signal path by blocking the binding of PD-1 to PD-L1. In order to determine the inhibition effect of the AmNB1613^{mutant}-FC on the binding of PD-1/PD-L1, the MOA detection system (PD-1/PD-L1 Block Bioassay, Cell Propagation Model, Catalog J1252) available from Promega, Inc. and a luciferase reporter MOA detection cell lines (Promega, CS187109) were used in this example. According to the method provided in the product manual, the activation of NFAT signal was reflected by detecting the expression of luciferase reporter genes, thereby detecting the inhibitory effects of AmNB1613^{mutant}-FC on the binding of PD-1 to PD-L1, and the specific steps were as follows:
1. Treatment of PD-L1⁺ CHOK1 cells: PD-L1⁺ CHOK1 cells (from the MOA detection system described above, i.e., PD-L1 aAPC/CHO-K1 cells: CHO-K1 cells stably expressing human PD-L1 and activating cell surface proteins of related TCRs in an antigen-independent manner) were plated the day before the activity assay, and were passaged 1 to 2 days before plating CHOK1-PDL1 cells; after culturing, the cells were washed with PBS (Gibco) once, then pancreatin (Gibco, 25200072) was added for digestion for 5 min at 37° C., 5% CO₂, and then the digestion was stopped with four-fold the volume of the cell mixture of RPMI1640 (Gibco, 22400-071) medium containing 10% FBS (HyClone, SH30084.03), and the cells were collected; a small amount of the cell mixtures was taken to determine the cell concentration, a needed volume of cell suspension was centrifuged at 230 g for 10 min to remove the supernatant, the cells were resuspended to 4×10⁵ cells/mL by using RPMI1640 medium containing 10% FBS, the cells were added into a white 96-well cell culture plate (Nunclon, 136101) at 100 μL/well, and PBS was added into edge wells at 200 L/well; the cells were incubated overnight in an incubator at 37° C./5% CO₂.
2. Treatment of Jurkat-PD1 cells (from the MOA detection system described above, i.e., PD-1 effector cells: Jurkat T cells expressing luciferase induced by the nuclear factor of activated T cells (NFAT) and stably expressing human PD-1): the cells were passaged two days before the activity assay, the cells were counted, a needed volume of cell suspension was taken and centrifuged at 170 g for 5 min, and the cells were resuspended to 1.3×10⁶ cells/mL with assay buffer (1640 medium+10% FBS).
3. Incubation: supernatant was discarded from the PD-L1⁺ CHOK1 cell plate in step 1, and 40 μL of different concentrations of candidate antibodies (4 AmNB1613^{mutant}-FCs, as well as HzNB1613 as a control, and IgG1 as a negative control (see SEQ ID NO: 41 and SEQ ID NO: 42)) and 40 μL of Jurkat-PD1 cells were added, wherein the final antibody concentration was diluted in a 3-fold gradient from a final concentration of 66 nM for 11 gradients, and the mixture was incubated in a incubator at 37° C./5% CO₂ for 6 h.
4. Detection: Bio-Glo™ buffer and Bio-Glo™ substrate in the kit (Promega, G7940) were mixed uniformly in advance, 80 μL of the mixture was added into the 96-well plate in the step 3, the mixture is incubated for 10 min at room temperature, and full-wavelength chemiluminescence was collected using a Spectra Max I3 microplate reader (Thermo, Max13), the collection time of each well was 1000 ms, and the experimental result is shown in FIG. 2.

It can be seen from FIG. 2, antibodies AmNB1613^{mutant}-FCs (AmNB1613.1, AmNB1613.12, AmNB1613.25, AmNB1613.28, and AmNB1613.36) all have higher NFAT signals than that of the parent antibody HzNB1613, indicating that the AmNB1613^{mutant}-FC is more effective in blocking the PD1/PD-L1 interaction.

### Example 7: Assay for Thermal Stability of AmNB1613^{mutant}-FC

With differential scanning fluorimetry (DSF), information about structure stability can be provided according to the process of fluorescence change in an atlas, and the configuration change of a protein can be detected. The temperature corresponding to the maximum absolute value of the fluorescence curve is the Tm of the protein. This study used differential scanning fluorescence (DSF) to detect the thermal stability of protein and assay the Tm values of AmNB1613^{mutant}-FCs (AmNB1613.1, AmNB1613.12, AmNB1613.25, AmNB1613.28, and AmNB1613.36), and the specific steps were as follows:
1. Purified antibody samples prepared as described above were diluted to 1 mg/mL with PBS; The SYPRO Orange protein gel stain (Gibco, S6650) was diluted 50-fold with PBS, i.e., 196 μL of PBS was added to 4 μL of the SYPRO Orange protein gel stain stock solution.
2. 50 μL of 1 mg/mL HzNB1613-FC as a control and AmNB1613^{mutant}-FC samples were added into a 96-well plate, and 10 μL of SYPRO Orange protein gel stain diluent and 40 μL of ddH₂O were added into each well.
3. The plate was placed in a 7500 real time PCR system for testing, and the results are shown in Table 6.

It can be seen from Table 6, the Tm values of AmNB1613.1, AmNB1613.12, AmNB1613.25, AmNB1613.28, and AmNB1613.36 are slightly lower than that of the parent antibodies, but all are greater than 54° C., and thus all of these antibodies have better thermal stability.

TABLE 6
Thermal stability
assay of AmNB1613^{Mutant}-FC

| Antibodies | Tm (° C.) | | | Average (° C.) |
|---|---|---|---|---|
| HzNB1613 | 62.97 | 63.16 | 62.97 | 63.03 |
| AmNB1613.1 | 61.45 | 61.26 | 61.26 | 61.32 |
| AmNB1613.12 | 58.79 | 58.79 | 58.6 | 58.73 |
| AmNB1613.25 | 61.07 | 61.26 | 61.45 | 61.26 |
| AmNB1613.28 | 58.41 | 58.41 | 58.41 | 58.41 |
| AmNB1613.36 | 54.05 | 54.24 | 54.24 | 54.18 |

### Example 8: Accelerated Stability Study of AmNB1613^{mutant}-FC

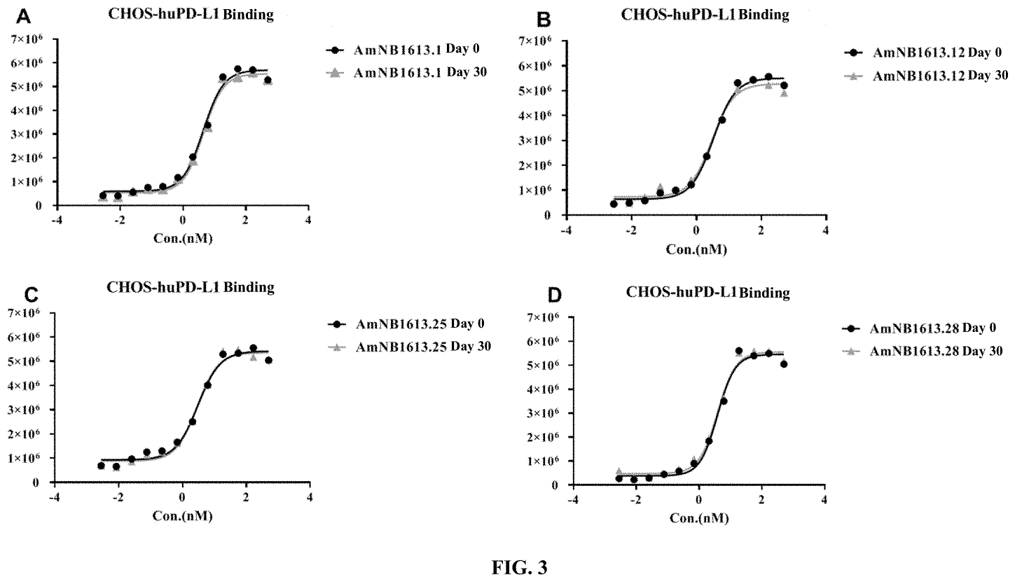

To further confirm the stability of the mutant antibody, in this example, changes in purity of the mutant antibody after placing at 40° C. for 0 and 30 days were assayed, and thus the long-term thermal stability of the antibody was evaluated. The method is specifically as follows:
1. The antibody samples obtained in Example 3 (AmNB1613.1, AmNB1613.12, AmNB1613.25 and AmNB1613.28) were concentrated to 10 mg/mL (in PBS), then aliquoted into EP tubes at 200 μL/tube and placed at 40° C. in the absence of light.
2. One tube was taken on day 0, 1, 3, 7, 10, 20 and 30, and the main peak purity of monomer thereof was assayed by SEC-HPLC. The results are shown in Table 7.
3. Similar to Example 5, samples (AmNB1613.1, AmNB1613.12, AmNB1613.25, and AmNB1613.28) in the accelerated stability study were also detected for binding activity to the cell surface antigen, and the results are shown in FIG. 3.

It can be seen from Table 7 and FIG. 3: 1) the antibodies AmNB1613.1, AmNB1613.12, AmNB1613.25, and AmNB1613.28 have no obvious change in the proportion of main peaks of monomers thereof after being placed at 40° C. for 30 days, indicating excellent thermal stability of these antibodies; 2) the antibodies have good stability and no influence on the binding activity to the antigen.

TABLE 7
Accelerated stability
study of AmNB1613^{mutant}-FC

| | Proportion of monomer | |
|---|---|---|
| | main peak (%) | |
| Antibodies | Day 0 | Day30 |
| AmNB1613.1 | 96.25 | 96.69 |
| AmNB1613.12 | 96.14 | 96.17 |
| AmNB1613.25 | 97.98 | 98.63 |
| AmNB1613.28 | 98.24 | 97.25 |

### Example 9: Solubility Study of AmNB1613^{mutant}-FC

In this study, the solubility of the antibody was reflected by the dissolution of the candidate antibodies (AmNB1613.1, AmNB1613.12, AmNB1613.25, and AmNB1613.28) in different concentrations of PEG, using the PEG precipitation method (Li et al., Application of a PEG precipitation method for solubility screening: A tool for developing high protein concentration formulations. Protein Science, 2013. 22: p. 1118-23) and with the Humira antibody as a positive control. The experimental processes are as follows:
1. Antibody samples (AmNB1613.1, AmNB1613.12, AmNB1613.25 and AmNB1613.28 as well as HzNB1613 and Humira, prepared as in Example 3) were concentrated to 5 mg/mL.
2. 40 μL of each antibody sample was taken and added to a 96-well plate, and wells in columns 1 to 12 were added with 30% PEG6000 (Sigma, 81255-250G) 13.4 μL, 26.7 L, 40.0 μL, 46.7 μL, 53.3 μL, 60.0 μL, 66.7 μL, 73.3 μL, 80.0 μL, 86.7 μL, 93.3 μL and 100.0 μL, and each well was replenished with PBS to a total volume of 200 μL; the final concentration of PEG of each well was 2%, 4%, 6%, 7%, 8%, 9%, 10%, 11%, 12%, 13%, 14% and 15%.
3. The 96-well plate was placed at room temperature for 1 h, and OD₅₀₀ nm was measured.

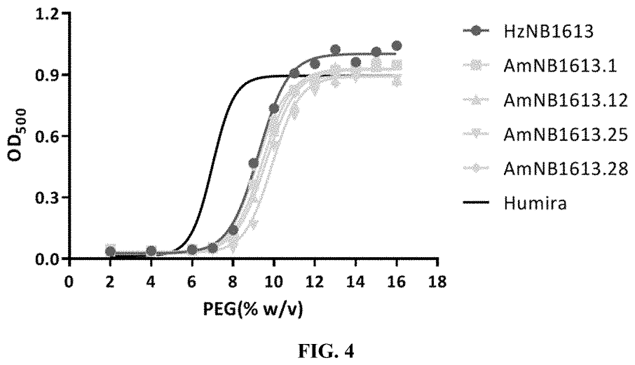

The experimental results are shown in FIG. 4, and the antibody of the present invention has better solubility than Humira, indicating that the antibody is suitable for the later drug development.

### Example 10: SPR Assay for Affinity of AmNB1613^{mutant}-FC

The equilibrium dissociation constant (K_{D}) of the antibody binding to human PD-L1 before and after affinity maturation according to the present invention was determined by surface plasmon resonance (SPR). Based on the principle of SPR, when a beam of polarized light entered the end face of a prism at a certain angle, surface plasma waves were generated at the interface between the prism and a gold film, causing free electrons in a metal film to generate resonance, namely surface plasmon resonance. When in analysis, a biomolecule recognition membrane was fixed on the surface of a sensing chip, then a sample to be detected flowed on the surface of the chip. If there were molecules capable of interacting with the biomolecule recognition membrane on the surface of the chip in the sample, the refractive index of a gold film surface was changed, finally causing changes in the SPR angle. The information such as the affinity and the kinetic constant of an analyte could be obtained by detecting the SPR angle changes.

The K_{D} was determined by a capture method, in which after the antibody was captured to the chip by the anti-human Fc antibody, affinity and kinetic constant were obtained by detecting the binding and dissociation between an antigen and the captured antibody. The method comprises chip preparation and affinity detection. The assay procedure used HBS-EP⁺ (BR-1006-69, GE Healthcare) diluted 10 times as an experimental buffer. The chip preparation process used an amino coupling kit (BR-1006-33, GE Healthcare) to couple an anti-human Fc antibody on the surface of a CM5 chip (29-1496-03, GE Healthcare), and the specific processes were as follows: first, 50 mM N-hydroxysuccinimide (NHS) and 200 mM 1-ethyl-3-(3-dimethylaminopropyl)carbodiimide hydrochloride (EDC) were freshly mixed and injected into a CM5 chip dual channel for activation for 7 min. Anti-human Fc antibodies were then diluted in 10 mM acetic acid (pH 5.0) and injected into the CM5 chip dual channel to covalently couple proteins to the chip channel surface at a coupling height of about 6000 RU. Finally, 1 M ethanolamine was injected and the remaining activation sites were blocked for 7 min.

Each cycle for affinity assay included capture of antibody, binding of one concentration of antigen, and chip regeneration:

Capture of antibody: the antibodies (HzNB1613, AmNB1613.1, AmNB1613.12, AmNB1613.25, and AmNB1613.28) prepared in Example 3 were first diluted to 0.5 μL/mL and captured at a flow rate of 10 μL/min for 30 s on the second channel of the CM5 chip.

Binding antigen: human PD-L1 (Acro Biosystems, Catalog No. PD1-H5229) was diluted with a two-fold gradient of assay buffer to between 0.15 nM and 20 nM according to the optimal range of SPR concentration, and injected into the CM5 chip dual channel in ascending order of concentration, bound for 180 s, and dissociated for 600 s.

Chip regeneration: the chips were regenerated using 10 mM Glycine pH 1.5 (BR-1003-54, GE Healthcare) before the next antibody assay was performed.

Kinetic analysis on the assay results was performed using a 1:1 binding model. In the assay described above, antibodies HzNB1613, AmNB1613.1, AmNB1613.12, AmNB1613.25, and AmNB1613.28 of the present invention have affinities for human PD-L1 as shown in Table 8.

TABLE 8
Determination of K_{D} of AmNB1613^{mutant}-FC by SPR

| Antibodies | ka (1/Ms) | kd (1/s) | KD (M) |
|---|---|---|---|
| HzNB1613 | 1.483E+6 | 0.005808 | 3.918E−9 |
| AmNB1613.1 | 1.731E+6 | 5.220E−4 | 3.015E−10 |
| AmNB1613.12 | 1.300E+6 | 5.311E−4 | 4.086E−10 |
| AmNB1613.25 | 2.399E+6 | 4.886E−4 | 2.037E−10 |
| AmNB1613.28 | 3.370E+6 | 5.947E−4 | 1.765E−10 |

It can be seen from the results in Table 8 that the affinity-matured mutant antibodies of the present invention have a 10-22 times higher affinity and excellent druggability.

SEQUENCE LISITNG
Related sequences of exemplary antibodies (where CDRs are defined
by Chothia rules)

| | HzNB1613 | | | |
|---|---|---|---|---|
| Antibody ID | (AmNB1613.0) | AmNB1613.1 | AmNB1613.12 | AmNB1613.25 |
| FW1 | QVQLQESGGG | SEQ ID NO: 9 | SEQ ID NO: 9 | SEQ ID NO: 9 |
| | LVQPGGSLRLS | | | |
| | CAAS (SEQ ID | | | |
| | NO: 9) | | | |
| CDR1 | AYTISRNSMG | ADPMSRNSMG | DNPMSRNSAG | SEQ ID NO: 1 |
| | (SEQ ID NO: 1) | (SEQ ID NO: 2) | (SEQ ID NO: 3) | |
| FW2 | WFRQAPGKGL | SEQ ID NO: 10 | SEQ ID NO: 10 | SEQ ID NO: 10 |
| | EGVA | | | |
| | (SEQ ID NO: 10) | | | |
| CDR2 | AIESDGSTS | SEQ ID NO: 4 | SEQ ID NO: 4 | SEQ ID NO: 4 |
| | (SEQ ID NO: 4) | | | |
| FW3 | YSDSVKGRFTI | SEQ ID NO: 11 | SEQ ID NO: 11 | SEQ ID NO: 11 |
| | SLDNSKNTLYL | | | |
| | EMNSLRAEDT | | | |
| | AVYYCAA | | | |
| | (SEQ ID NO: 11) | | | |
| CDR3 | PKVGLGPRTAL | SEQ ID NO: 5 | SEQ ID NO: 5 | PKVGLGPRTAL |
| | GHLAFMTLPA | | | GHLAFMTLQD |
| | LNY | | | LND |
| | (SEQ ID NO: 5) | | | (SEQ ID NO: 6) |
| FW4_VH | WGQGTLVTVSS | SEQ ID NO: 12 | SEQ ID NO: 12 | SEQ ID NO: 12 |
| | (SEQ ID | | | |
| | NO: 12) | | | |
| V_{H}H (or heavy | QVQLQESGGG | QVQLQESGGG | QVQLQESGGG | QVQLQESGGG |
| chain variable | LVQPGGSLRLS | LVQPGGSLRLS | LVQPGGSLRLS | LVQPGGSLRLS |
| region VH) | CAASAYTISRN | CAASADPMSR | CAASDNPMSR | CAASAYTISRN |
| | SMGWFRQAPG | NSMGWFRQAP | NSAGWFRQAP | SMGWFRQAPG |
| | KGLEGVAAIES | GKGLEGVAAIE | GKGLEGVAAIE | KGLEGVAAIES |
| | DGSTSYSDSVK | SDGSTSYSDSV | SDGSTSYSDSV | DGSTSYSDSVK |
| | GRFTISLDNSK | KGRFTISLDNS | KGRFTISLDNS | GRFTISLDNSK |
| | NTLYLEMNSL | KNTLYLEMNS | KNTLYLEMNS | NTLYLEMNSL |
| | RAEDTAVYYC | LRAEDTAVYY | LRAEDTAVYY | RAEDTAVYYC |
| | AAPKVGLGPR | CAAPKVGLGP | CAAPKVGLGP | AAPKVGLGPR |
| | TALGHLAFMT | RTALGHLAFM | RTALGHLAFM | TALGHLAFMT |
| | LPALNYWGQG | TLPALNYWGQ | TLPALNYWGQ | LQDLNDWGQG |
| | TLVTVSS (SEQ | GTLVTVSS (SEQ | GTLVTVSS(SEQ | TLVTVSS(SEQ |
| | ID NO: 13) | ID NO: 14) | ID NO: 15) | ID NO: 16) |
| Heavy-chain | QVQLQESGGG | QVQLQESGGG | QVQLQESGGG | QVQLQESGGG |
| antibody | LVQPGGSLRLS | LVQPGGSLRLS | LVQPGGSLRLS | LVQPGGSLRLS |
| | CAASAYTISRN | CAASADPMSR | CAASDNPMSR | CAASAYTISRN |
| | SMGWFRQAPG | NSMGWFRQAP | NSAGWFRQAP | SMGWFRQAPG |
| | KGLEGVAAIES | GKGLEGVAAIE | GKGLEGVAAIE | KGLEGVAAIES |
| | DGSTSYSDSVK | SDGSTSYSDSV | SDGSTSYSDSV | DGSTSYSDSVK |
| | GRFTISLDNSK | KGRFTISLDNS | KGRFTISLDNS | GRFTISLDNSK |
| | NTLYLEMNSL | KNTLYLEMNS | KNTLYLEMNS | NTLYLEMNSL |
| | RAEDTAVYYC | LRAEDTAVYY | LRAEDTAVYY | RAEDTAVYYC |
| | AAPKVGLGPR | CAAPKVGLGP | CAAPKVGLGP | AAPKVGLGPR |
| | TALGHLAFMT | RTALGHLAFM | RTALGHLAFM | TALGHLAFMT |
| | LPALNYWGQG | TLPALNYWGQ | TLPALNYWGQ | LQDLNDWGQG |
| | TLVTVSSDKTH | GTLVTVSSDKT | GTLVTVSSDKT | TLVTVSSDKTH |
| | TCPPCPAPEAA | HTCPPCPAPEA | HTCPPCPAPEA | TCPPCPAPEAA |
| | GGPSVFLFPPK | AGGPSVFLFPP | AGGPSVFLFPP | GGPSVFLFPPK |
| | PKDTLMISRTP | KPKDTLMISRT | KPKDTLMISRT | PKDTLMISRTP |
| | EVTCVVVDVS | PEVTCVVVDV | PEVTCVVVDV | EVTCVVVDVS |
| | HEDPEVKFNW | SHEDPEVKFN | SHEDPEVKFN | HEDPEVKFNW |
| | YVDGVEVHNA | WYVDGVEVH | WYVDGVEVH | YVDGVEVHNA |
| | KTKPREEQYNS | NAKTKPREEQ | NAKTKPREEQ | KTKPREEQYNS |
| | TYRVVSVLTV | YNSTYRVVSV | YNSTYRVVSV | TYRVVSVLTV |
| | LHQDWLNGKE | LTVLHQDWLN | LTVLHQDWLN | LHQDWLNGKE |
| | YKCKVSNKAL | GKEYKCKVSN | GKEYKCKVSN | YKCKVSNKAL |
| | PAPIEKTISKAK | KALPAPIEKTIS | KALPAPIEKTIS | PAPIEKTISKAK |
| | GQPREPQVYTL | KAKGQPREPQ | KAKGQPREPQ | GQPREPQVYTL |
| | PPSRDELTKNQ | VYTLPPSRDEL | VYTLPPSRDEL | PPSRDELTKNQ |
| | VSLTCLVKGFY | TKNQVSLTCL | TKNQVSLTCL | VSLTCLVKGFY |
| | PSDIAVEWESN | VKGFYPSDIAV | VKGFYPSDIAV | PSDIAVEWESN |
| | GQPENNYKTT | EWESNGQPEN | EWESNGQPEN | GQPENNYKTT |
| | PPVLDSDGSFF | NYKTTPPVLDS | NYKTTPPVLDS | PPVLDSDGSFF |
| | LYSKLTVDKSR | DGSFFLYSKLT | DGSFFLYSKLT | LYSKLTVDKSR |
| | WQQGNVFSCS | VDKSRWQQGN | VDKSRWQQGN | WQQGNVFSCS |
| | VMHEALHNHY | VFSCSVMHEA | VFSCSVMHEA | VMHEALHNHY |
| | TQKSLSLSPGK | LHNHYTQKSL | LHNHYTQKSL | TQKSLSLSPGK |
| | (SEQ ID | SLSPGK (SEQ | SLSPGK (SEQ | (SEQ ID NO: 22) |
| | NO: 19) | ID NO: 20) | ID NO: 21) | |
| | | | | Consensus |
| | Antibody ID | AmNB1613.28 | AmNB1613.36 | sequences |
| | FW1 | SEQ ID NO: 9 | SEQ ID NO: 9 | SEQ ID NO: 9 |
| | CDR1 | SEQ ID NO: 1 | SEQ ID NO: 1 | X₁X₂X₃X₄SRNS |
| | | | | X₅G, wherein X₁ |
| | | | | may be A or D, |
| | | | | X₂ may be Y or |
| | | | | D or N; X₃ may |
| | | | | be T or P, X₄ may |
| | | | | be I or M, and X₅ |
| | | | | may be M or A |
| | | | | (SEQ ID NO: 44) |
| | FW2 | SEQ ID NO: 10 | SEQ ID NO: 10 | SEQ ID NO: 10 |
| | CDR2 | SEQ ID NO: 4 | SEQ ID NO: 4 | SEQ ID NO: 4 |
| | FW3 | SEQ ID NO: 11 | SEQ ID NO: 11 | SEQ ID NO: 11 |
| | CDR3 | PKVGLGPRTAL | PKVGLGPRTAL | AAPKVGLGPR |
| | | GHLAFMILQDL | GHLAFMILADL | TALGHLAFMX₁ |
| | | NY | ND | LX₂X₃LNX₄, |
| | | (SEQ ID NO: 7) | (SEQ ID NO: 8) | wherein X₁ may |
| | | | | be T or I, X₂ may |
| | | | | be P or Q or A, |
| | | | | X₃ may be A or |
| | | | | D, and X₄ may be |
| | | | | Y or D |
| | | | | (SEQ ID NO: 45) |
| | FW4_VH | SEQ ID NO: 12 | SEQ ID NO: 12 | SEQ ID NO: 12 |
| | V_{H}H (or heavy | QVQLQESGGG | QVQLQESGGG | |
| | chain variable | LVQPGGSLRLS | LVQPGGSLRLS | |
| | region VH) | CAASAYTISRN | CAASAYTISRN | |
| | | SMGWFRQAPG | SMGWFRQAPG | |
| | | KGLEGVAAIES | KGLEGVAAIES | |
| | | DGSTSYSDSVK | DGSTSYSDSVK | |
| | | GRFTISLDNSK | GRFTISLDNSK | |
| | | NTLYLEMNSL | NTLYLEMNSL | |
| | | RAEDTAVYYC | RAEDTAVYYC | |
| | | AAPKVGLGPR | AAPKVGLGPR | |
| | | TALGHLAFMIL | TALGHLAFMIL | |
| | | QDLNYWGQGT | ADLNDWGQGT | |
| | | LVTVSS(SEQ | LVTVSS(SEQ | |
| | | ID NO: 17) | ID NO: 18) | |
| | Heavy-chain | QVQLQESGGG | QVQLQESGGG | |
| | antibody | LVQPGGSLRLS | LVQPGGSLRLS | |
| | | CAASAYTISRN | CAASAYTISRN | |
| | | SMGWFRQAPG | SMGWFRQAPG | |
| | | KGLEGVAAIES | KGLEGVAAIES | |
| | | DGSTSYSDSVK | DGSTSYSDSVK | |
| | | GRFTISLDNSK | GRFTISLDNSK | |
| | | NTLYLEMNSL | NTLYLEMNSL | |
| | | RAEDTAVYYC | RAEDTAVYYC | |
| | | AAPKVGLGPR | AAPKVGLGPR | |
| | | TALGHLAFMIL | TALGHLAFMIL | |
| | | QDLNYWGQGT | ADLNDWGQGT | |
| | | LVTVSSDKTHT | LVTVSS | |
| | | CPPCPAPEAAG | DKTHTCPPCPA | |
| | | GPSVFLFPPKP | PEAAGGPSVFL | |
| | | KDTLMISRTPE | FPPKPKDTLMI | |
| | | VTCVVVDVSH | SRTPEVTCVVV | |
| | | EDPEVKFNWY | DVSHEDPEVKF | |
| | | VDGVEVHNAK | NWYVDGVEV | |
| | | TKPREEQYNST | HNAKTKPREE | |
| | | YRVVSVLTVL | QYNSTYRVVS | |
| | | HQDWLNGKEY | VLTVLHQDWL | |
| | | KCKVSNKALP | NGKEYKCKVS | |
| | | APIEKTISKAK | NKALPAPIEKTI | |
| | | GQPREPQVYTL | SKAKGQPREP | |
| | | PPSRDELTKNQ | QVYTLPPSRDE | |
| | | VSLTCLVKGFY | LTKNQVSLTCL | |
| | | PSDIAVEWESN | VKGFYPSDIAV | |
| | | GQPENNYKTT | EWESNGQPEN | |
| | | PPVLDSDGSFF | NYKTTPPVLDS | |
| | | LYSKLTVDKSR | DGSFFLYSKLT | |
| | | WQQGNVFSCS | VDKSRWQQGN | |
| | | VMHEALHNHY | VFSCSVMHEA | |
| | | TQKSLSLSPGK | LHNHYTQKSL | |
| | | (SEQ ID NO: 23) | SLSPGK (SEQ | |
| | | | ID NO: 24) | |

SEQ ID NO: 25: amino acid sequence of Fc region of human IgG1 LALA
SEQ ID NO: 26: sequence of pYDC011 plasmid
Primer (5′-3′):
SEQ ID NO: 27: AMP0044; SEQ ID NO: 28: AMP0082; SEQ ID NO: 29: AMP0083;
SEQ ID NO: 30: AMP0084;
SEQ ID NO: 31: AMP0085; SEQ ID NO: 32: AMP0086; SEQ ID NO: 33: AMP0087;
SEQ ID NO: 34: AMP0088;
SEQ ID NO: 35: AMP0089; SEQ ID NO: 36: AMP0090; SEQ ID NO: 37: AMP0091;
SEQ ID NO: 38: AMP0092;
SEQ ID NO: 39: AMP0093; SEQ ID NO: 40: AMP0094;
SEQ ID NO: 41: heavy chain (HC) amino acid sequence of IgG1 negative control:
SEQ ID NO: 42: light chain (LC) amino acid sequence of IgG1 negative control.

1. An antibody or antigen-binding fragment thereof that binds to PD-L1, wherein the antibody comprises a heavy chain variable region VH and optionally an Fc region, and the VH comprises 3 complementarity determining regions (CDRs) contained in a VH set forth in any one of SEQ ID NOs: 14 to 18.

2. An antibody or antigen-binding fragment thereof that binds to PD-L1, wherein the antibody comprises a VH and optionally an Fc region, and the VH comprises 3 complementarity determining regions HCDRs of a heavy chain variable region, wherein
(i) HCDR1 comprises or consists of an amino acid sequence set forth in SEQ ID NO: 2, HCDR2 comprises or consists of an amino acid sequence set forth in SEQ ID NO: 4, and HCDR3 comprises or consists of an amino acid sequence set forth in SEQ ID NO: 5;
(ii) the HCDR1 comprises or consists of an amino acid sequence set forth in SEQ ID NO: 3, the HCDR2 comprises or consists of an amino acid sequence set forth in SEQ ID NO: 4, and the HCDR3 comprises or consists of an amino acid sequence set forth in SEQ ID NO: 5;
(iii) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 6;
(iv) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 7; or
(v) the HCDR1 comprises or consists of an amino acid sequence set forth in SEQ ID NO: 1, the HCDR2 comprises or consists of an amino acid sequence set forth in SEQ ID NO: 4, and the HCDR3 comprises or consists of an amino acid sequence set forth in SEQ ID NO: 8.

3. The antibody or the antigen-binding fragment thereof according to any one of claim 1 or 2, wherein the antibody comprises a heavy chain variable region VH and optionally an Fc region, and the VH comprises or consists of an amino acid sequence having at least 90% identity to an amino acid sequence selected from any one of SEQ ID NOs: 14 to 18, or comprises or consists of an amino acid sequence selected from any one of SEQ ID NOs: 14 to 18.

4. The antibody or the antigen-binding fragment thereof according to any one of claim 1 or 2, wherein the Fc region is from an IgG, e.g., IgG1, IgG2, IgG3, or IgG4.

5. The antibody or the antigen-binding fragment thereof according to any one of claim 1 or 2, wherein the antibody comprises or consists of a heavy chain, and the heavy chain comprises or consists of an amino acid sequence having at least 85% identity to an amino acid sequence selected from any one of SEQ ID NOs: 20 to 24, or the heavy chain comprises or consists of an amino acid sequence selected from any one of SEQ ID NOs: 20 to 24.

6. The antibody or the antigen-binding fragment thereof that binds to PD-L1 according to any one of claims 1 to 5, wherein the antibody is a single-domain antibody or a heavy-chain antibody.

7. The antibody or the antigen-binding fragment thereof that binds to PD-L1 according to any one of claims 1 to 6, having one or more of the following features:
(1) binding to PD-L1 (e.g., human PD-L1) with high affinity, e.g., binding to human PD-L1 with a K_{D} of less than or equal to 0.5 nM as determined by surface plasmon resonance (SPR);
(2) having a lower dissociation rate, e.g., less than or equal to about 6×10⁻⁴ l/s as determined by surface plasmon resonance (SPR) after binding to PD-L1;
(3) binding to a cell expressing human PD-L1, e.g., with an EC₅₀ of less than or equal to about 7.5 nM;
(4) blocking the relevant activity of PD-L1;
(5) having good thermal stability and/or solubility and/or druggability;
(6) having a high expression yield;
(7) exhibiting the same or similar binding affinity and/or specificity for PD-L1 as any one of the antibodies according to claim 5;
(8) inhibiting (e.g., competitively inhibiting) the binding of any one of the antibodies according to claim 5 to PD-L1;
(9) binding to the same or overlapping epitope as antibodies according to claim 5;
(10) competing with the antibodies according to claim 5 for binding to PD-L1; and
(11) having one or more biological properties of the antibodies according to claim 5.

8. The anti-PD-L1 antibody or the antigen-binding fragment thereof according to any one of claims 1 to 7, wherein the antibody is a humanized antibody, a human antibody, or a chimeric antibody.

9. The antibody or the antigen-binding fragment thereof according to any one of claims 1 to 11, wherein the antibody is a bispecific or multispecific antibody.

10. An isolated nucleic acid encoding the anti-PD-L1 antibody or the fragment thereof according to any one of claims 1 to 9.

11. A vector comprising the nucleic acid according to claim 10, wherein, preferably, the vector is an expression vector, such as a pTT5 vector.

12. A host cell comprising the nucleic acid according to claim 10 or the vector according to claim 11, wherein, preferably, the host cell is prokaryotic or eukaryotic, more preferably, the host cell is selected from E. coli cells, yeast cells, mammalian cells, and other cells suitable for preparation of the antibody or the antigen-binding fragment thereof, and most preferably, the host cell is a 293 cell or a CHO cell.

13. A method for preparing an anti-PD-L1 antibody or the antigen-binding fragment thereof, wherein the method comprises incubating the host cell according to claim 12 in conditions suitable for expression of a nucleic acid encoding the anti-PD-L1 antibody or the antigen-binding fragment thereof according to any one of claims 1 to 9, and optionally, isolating the antibody or the antigen-binding fragment thereof; optionally the method further comprises recovering the anti-PD-L1 antibody or the antigen-binding fragment thereof from the host cell.

14. An immunoconjugate, comprising the anti-PD-L1 antibody or the antigen-binding fragment thereof according to any one of claims 1 to 9, and an additional substance.

15. A pharmaceutical composition, comprising the anti-PD-L1 antibody or the antigen-binding fragment thereof according to any one of claims 1 to 9 or the immunoconjugate according to claim 14, and optionally a pharmaceutical excipient.

16. A pharmaceutical composition, comprising the anti-PD-L1 antibody or the antigen-binding fragment thereof according to any one of claims 1 to 9 or the immunoconjugate according to claim 14 and other therapeutic agents, and optionally a pharmaceutical excipient; wherein, preferably, the other therapeutic agents are selected from chemotherapeutic agents, other antibodies, cytotoxic agents, vaccines, anti-infective active agents, small molecule drugs, or immunomodulatory agents.

17. A combination product, comprising the antibody or the antigen-binding fragment thereof that binds PD-L1 according to any one of claims 1 to 9 or the immunoconjugate according to claim 14, and one or more other therapeutic agents, such as chemotherapeutic agents, cytotoxic agents, vaccines, other antibodies, anti-infective active agents, small molecule drugs, or immunomodulatory agents.

18. A method for preventing or treating a tumor or an infectious disease in a subject or an individual, comprising administering to the subject an effective amount of the anti-PD-L1 antibody or the antigen-binding fragment thereof according to any one of claims 1 to 9, or the immunoconjugate according to claim 14, or the pharmaceutical composition according to claim 15 or 16, or the combination product according to claim 17.

19. The method according to claim 18, wherein the tumor is a cancer, such as a cancer with elevated expression levels of PD-1, PD-L1, or PD-L2.

20. The method according to any one of claim 18 or 19, wherein the antibody or the antigen-binding fragment thereof or the immunoconjugate thereof is further capable of being administered in combination with one or more other therapies, such as therapeutic modalities and/or additional therapeutic agents, preferably, the therapeutic modalities comprise surgical treatment and/or radiation therapy, or the therapeutic agents are selected from chemotherapeutic agents, cytotoxic agents, vaccines, anti-infective active agents, other antibodies, small molecule drugs, or immunomodulatory agents.

21. A method for detecting PD-L1 in a sample, comprising
(a) contacting a sample with the anti-PD-L1 antibody or the antigen-binding fragment thereof according to any one of claims 1 to 9; and
(b) detecting the formation of a complex by the anti-PD-L1 antibody or the antigen-binding fragment thereof and PD-L1, wherein, optionally, the anti-PD-L1 antibody is detectably labeled.
